(19) United States
(12) Patent Application Publication
(10) Pub. No.: US 2003/0013739 A1
(43) Pub. Date: Jan. 16, 2003

(54) Methods of using a combination of cyclooxygenase-2 selective inhibitors and thalidomide for the treatment of neoplasia

(75) Inventor: Jaime L. Masferrer, Ballwin, MO (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Pharmacia Corporation

(21) Appl. No.: 10/135,793

(22) Filed: Apr. 30, 2002

Related U.S. Application Data
(63) Continuation-in-part of application No. 09/470,951, filed on Dec. 22, 1999.
(60) Provisional application No. 60/113,786, filed on Dec. 23, 1998.

(51) Int. Cl. A61K 31/454; A61K 31/415

(52) U.S. Cl. 514/323; 514/406

(57) ABSTRACT

The present invention provides compositions and methods for the treatment, prevention or inhibition of neoplasia by administering an effective amount of a cyclooxygenase-2 selective inhibitor in combination with an effective amount of thalidomide.

This application is a continuation-in-part application of PCT/US99/30693, filed Dec. 22, 1999, which claims priority from U.S. provisional patent application no. 60/113,786, filed Dec. 23, 1998, both of which are incorporated herein by reference.

### BACKGROUND

### Field of the Invention

The present invention is directed to compositions comprising a cyclooxygenase-2 inhibitor or a pharmaceutically acceptable salt, ester or prodrug thereof and a thalidomide, thalidomide analog, thalidomide hydrolysis product, thalidomide metabolite or thalidomide precursor, wherein said compositions are useful for the treatment, prevention or inhibition of neoplasia disorder. Further provided are methods for treatment, prevention or inhibition of neoplasia disorders utilizing said compositions.

Cancer is now the second leading cause of death in the United States where over 8,000,000 persons have been diagnosed with some form of cancer. In 1995, cancer accounted for 23.3% of all deaths in the United States. (See U.S. Dept. of Health and Human Services, National Center for Health Statistics, Health United States 1996-97 and Injury Chartbook 117 (1997)).

Cancer is not fully understood on the molecular level. It is known that exposure of a cell to a carcinogen such as certain viruses, chemicals, or radiation, leads to DNA alteration that inactivates a “suppressive” gene or activates an “oncogene”. Suppressive genes are growth regulatory genes, which upon mutation, can no longer control cell growth. Oncogenes are initially normal genes (called protooncogenes) that by mutation or altered context of expression become transforming genes. The products of transforming genes cause inappropriate cell growth. More than twenty different normal cellular genes can become oncogenes by genetic alteration. Transformed cells differ from normal cells in many ways, including cell morphology, cell-to-cell interactions, membrane content, cytoskeletal structure, protein secretion, gene expression and mortality (transformed cells can grow indefinitely).

A neoplasm, or tumor, is an abnormal, unregulated, and disorganized proliferation of cell growth. A neoplasm is malignant, or cancerous, if it has properties of destructive growth, invasiveness and metastasis. Invasiveness refers to the local spread of a neoplasm by infiltration of destruction of surrounding tissue, typically breaking through the basal laminas that define the boundaries of the tissues, thereby often entering the body's circulatory system.

Metastasis typically refers to the dissemination of tumor cells via lymphatics or blood vessels. Metastasis also refers to the migration of tumor cells by direct extension through serous cavities, or subarachnoid or other spaces. Through the process of metastasis, tumor cell migration to other areas of the body establishes neoplasms in areas away from the site of initial appearance.

Angiogenesis is prominent in solid tumor formation and metastasis. Angiogenic factors have been found associated with several solid tumors such as rhabdomyosarcomas, retinoblastoma, Ewing sarcoma, neuroblastoma, and osteosarcoma. A tumor cannot expand without a blood supply to provide nutrients and remove cellular wastes. Tumors in which angiogenesis is important include solid tumors, and benign tumors such as acoustic neuroma, neurofibroma, trachoma and pyogenic granulomas. Angiogenesis has been associated with blood-born tumors such as leukemias, any of various acute or chronic neoplastic diseases of the bone marrow in which unrestrained proliferation of white blood cells occurs, usually accompanied by anemia, impaired blood clotting, and enlargement of the lymph nodes, liver, and spleen. It is believed that angiogenesis plays a role in the abnormalities in the bone marrow that give rise to leukemia-like tumors. Prevention of angiogenesis could halt the growth of cancerous tumors and the resultant damage to the animal due to the presence of the tumor. U.S. Pat. No. 5,843,925 describes a method for inhibiting angiogenesis and endothelial cell proliferation using a 7-[substituted amino]-9-[(substituted glycyl)amido]-6-demethyl-6-deoxytetracycline. U.S. Pat. No. 5,861,372 describes the use of an aggregate endothelial inhibitor, angiostatin, and its use in inhibiting angiogenesis. U.S. Pat. No. 5,885,795 describes method and compositions for treating diseases mediated by undesired and uncontrolled angiogenesis by administering purified angiostatin or angiostain derivatives. PCT/GB97/00650 describes the use of cinnoline derivatives for use in the production of an antiangiogenic and/or vascular permeability reducing effect. PCT/US97/09610 describes administration of an antiendogin monoclonal antibody, or fragments thereof, which is conjugated to at least one angiogenesis inhibitor or antitumor agent for use in treating tumor and angiogenesis-associated diseases. PCT/US97/20116 and U.S. Pat. No. 6,235,756 describe compositions and methods for inhibition of angiogenesis using thalidomide.

Thalidomide was first synthesized and marketed in the 1950's as a sedative. In 1961, administration of thalidomide to pregnant women resulted in an epidemic of congenial malformations. As a result, thalidomide was removed from the market.

However, further studies of thalidomide have shown that thalidomide can be safely used to treat a number of diseases, such as graft vs host disease (Lim et al., Lancet, 1:117, 1988), ulceritive colitis (Waters et al., Brit. Med. J., 1:792, 1979), and aphthous ulcers (Jenkins et al., Lancet, 2:1424-6, 1984). Thalidomide has been shown to inhibit TNF-alpha production in erythema nodosum leprosum patients (Sarno et al., 1991) and in vitro stimulated monocytes (Sampaio et al., J. Exp. Med., 173:699-703, 1991). Shannon et al. (Amer. Soc. for Microbiology Ann. Meeting, Abst. U-53, 1990) indicated that thalidomide inhibited IL-1 beta production in vitro. Furthermore, D'Amato et al. (Proc. Natl. Acad. Sci. (USA), 91:4082-5, 1994) demonstrated that thalidomide was an effective inhibitor of angiogenesis induced by bFGF in the rabbit cornea micropocket assay. Thus, the ability of thalidomide to exert inhibitory activity on angiogenesis-related cytokines such as IL-1 beta, TNF-alpha and bFGF suggested that thalidomide could be used as an anti-angiogenic agent.

Cancer is now primarily treated with one or a combination of three types of therapies: surgery, radiation, and chemotherapy. Surgery involves the bulk removal of diseased tissue. While surgery is sometimes effective in removing tumors located at certain sites, for example, in the breast, colon, and skin, it cannot be used in the treatment of tumors located in other areas, such as the backbone or in the treatment of disseminated neoplastic conditions such as leukemia.

Chemotherapy involves the disruption of cell replication or cell metabolism. It is used most often in the treatment of breast, lung, and testicular cancer. However, the adverse effects of systemic chemotherapy used in the treatment of neoplastic disease is most feared by patients undergoing treatment for cancer. Of these adverse effects nausea and vomiting are the most common and severe side effects. Other adverse side effects include cytopenia, infection, cachexia, mucositis in patients receiving high doses of chemotherapy with bone marrow rescue or radiation therapy; alopecia (hair loss); cutaneous complications (see M. D. Abeloff, et al: Alopecia and Cutaneous Complications. P. 755-56. In Abeloff, M. D., Armitage, J. O. Lichter, A. S., and Niederhuber, J. E. (eds) Clinical Oncology. Churchill Livingston, N.Y., 1992, for cutaneous reactions to chemotherapy agents), such as pruritis, urticaria, and angioedema; neurological complications; pulmonary and cardiac complications in patients receiving radiation or chemotherapy; and reproductive and endocrine complications.

Chemotherapy-induced side effects significantly impact the quality of life of the patient and may dramatically influence patient compliance with treatment.

Additionally, adverse side effects associated with chemotherapeutic agents are generally the major dose-limiting toxicity (DLT) in the administration of these drugs. For example, mucositis, is one of the major dose limiting toxicities for several anticancer agents, including the antimetabolite cytotoxic agents 5-FU, methotrexate, and antitumor antibiotics, such as doxorubicin. Many of these chemotherapy-induced side effects are severe, may lead to hospitalization, or require treatment with analgesics for the treatment of pain.

The adverse side effects induced by chemotherapeutic agents and radiation therapy have become of major importance to the clinical management of cancer patients.

Prostaglandins are arachidonate metabolites produced in virtually all mammalian tissues and possess diverse biologic capabilities, including vasoconstriction, vasodilation, stimulation or inhibition of platelet aggregation, and immunomodulation, primarily immunosupression (Moskowitz and Coughlins, Stroke 1981; 12: 882-86; Leung and Mihich. Nature 1980; 597-600; Brunda et al., J. Immunol. 1980; 124: 2682-7). Further, prostaglandins are implicated in the promotion of development and growth of malignant tumors (Honn et al., Prostaglandins 1981;21:833-64; Furuta et al., Cancer Res. 1989, 48, 3002-7; Taketo; J. Natl. Cancer Inst. 1998, 90, 1609-20). They are also involved in the response of tumor and normal tissues to cytotoxic agents such as ionizing radiation (Milas and Hanson, Eur. J. Cancer 1995, 31A, 1580-5). Prostaglandin production is mediated by two cyclooxygenase enzymes: Cox-1 and Cox-2. Cyclooxygenase-1 (Cox-1) is constitutively expressed and is ubiquitous. Cyclooxygenase-2 (Cox-2) is induced by diverse inflammatory stimuli (Isakson et al., Adv. Pros. Throm. Leuk Res. 1995, 23, 49-54).

Prostaglandin-mediated effects at both the microenvironmental and cellular levels have been implicated in the modulation of such response. Prostaglandin E₂, and prostaglandin I₂ protect jejunum crypt cells, and prostaglandin I₂ protects B16 melanoma cells from radiation damage. Inhibition of prostaglandin synthesis also induces an accumulation of cells in the G₂+M phases of the cell cycle, which are generally considered to be the most sensitive to ionizing radiation. With the inhibition of prostaglandin synthesis, prostaglandin-induced immunosuppressive activity was diminished and antitumor immunologic responses were able to potentiate tumor response to radiation. Finally, prostaglandins are vasoactive agents and are thus likely to regulate tumor blood flow and perfusion.

Studies indicate that prostaglandins synthesized by cyclooxygenases play a critical role in the initiation and promotion of cancer. Aberrant Cox-2 expression was first reported in colorectal carcinomas and adenomas, and has now been detected in various human cancers, including those of the breast. Moreover, Cox-2 is overexpressed in neoplastic lesions of the colon, breast, lung, prostate, esophagus, pancreas, intestine, cervix, ovaries, urinary bladder and head and neck. Cox-2 overexpression in murine mammary glands is sufficient to cause tumor formation. See Howe et al., Endocr. Relat. Cancer (2001) 8(2):97-114. In several in vitro and animal models, Cox-2 inhibitors have inhibited tumor growth and metastasis.

In addition to cancers per se, Cox-2 is also expressed in the angiogenic vasculature within and adjacent to hyperplastic and neoplastic lesions indicating that Cox-2 plays a role in angiogenesis. In both the mouse and rat, Cox-2 inhibitors markedly inhibited bFGF-induced neovascularization. The utility of Cox-2 inhibitors as chemopreventive, antiangiogenic and chemotherapeutic agents is described in the literature (Koki et al., Potential utility of Cox-2 inhibitors in chemoprevention and chemotherapy, Exp. Opin, Invest. Drugs (1999) 8(10) pp. 1623-1638, hereby incorporated by reference).

Nonsteroidal anti-inflammatory drugs (NSAIDs) non-selectively inhibit both cyclooxygenase enzymes and consequently can prevent, inhibit, or abolish the effects of prostaglandins. Increasing evidence shows that NSAIDs can inhibit the development of cancer in both experimental animals and in humans, can reduce the size of established tumors, and can increase the efficacy of cytotoxic cancer chemotherapeutic agents. Our own investigations have demonstrated that indomethacin prolongs tumor growth delay and increases the tumor cure rate in mice after radiotherapy (Milas et al., Cancer Res. 1990, 50, 4473-7). The influence of oxyphenylbutazone and radiation therapy on cervical cancer has been studied. (Weppelmann and Monkemeier, Gyn. Onc., 1984, 47, 196-9). However, treatment with NSAIDs are limited by toxicity to normal tissue, particularly by ulcerations and bleeding in the gastrointestinal tract, ascribed to the inhibition of Cox-1. Recently developed selective Cox-2 inhibitors exert potent anti-inflammatory activity but cause fewer side effects. Compounds which selectively inhibit cyclooxygenase-2 have been described in U.S. Pat. Nos. 5,380,738; 5,344,991; 5,393,790; 5,434,178; 5,474,995; 5,510,368 and WO documents WO96/06840, WO96/03388, WO96/03387, WO96/19469, WO96/25405, WO95/15316, WO94/15932, WO94/27980, WO95/00501, WO94/13635, WO94/20480, and WO94/26731.

Additional Cox-2 inhibitors have been described for the treatment of cancer (WO98/16227) and for the treatment of tumors (EP 927,555). Celecoxib, a specific inhibitor of Cox-2, exerted a potent inhibition of fibroblast growth factor-induced corneal angiogenesis in rats. (Masferrer et al., Proc. Am. Assoc. Cancer Research 1999, 40, 396).

Several Cox-2 inhibitors have been described for the treatment of cancer, tumors and neoplasia. FR 27 71 005 describes compositions containing a cyclooxygenase-2 inhibitor and N-methyl-d-asparate (NMDA) antagonist used to treat cancer and other diseases. WO 99/18960 describes a combination comprising a cyclooxygenase-2 inhibitor (iNOS) that can be used to treat colorectal and breast cancer. WO 98/41511 describes 5-(4-sulphunyl-phenyl)-pyridazinone derivatives used for treating cancer. WO 98/41516 describes (methylsulphonyl)phenyl-2-(5H)-furanone derivatives that can be used in the treatment of cancer. WO 98/16227 describes the use of cyclooxygenase-2 inhibitors in the treatment or prevention of neoplasia. WO 97/36497 describes a combination comprising a cyclooxygenase-2 inhibitor and a 5-lipoxygenase inhibitor useful in treating cancer. WO 97/11701 describes a combination comprising of a cyclooxygenase-2 inhibitor and a leukotriene B4 receptor antagonist useful in treating colorectal cancer. WO 97/29774 describes the combination of a cyclooxygenase-2 inhibitor and protstagladin or antiulcer agent useful in treating cancer. WO 96/03385 describes 3,4,-Di substituted pyrazole compounds given alone or in combination with NSAIDs, steroids, 5-LO inhibitors, LTB4 antagonists, or LTA4 hydrolase inhibitors that may be useful in the treatment of cancer. WO 98/16227 describes a method of using cyclooxygenase-2 inhibitors in the treatment and prevention of neoplasia.

Cox-2 specific inhibitors prevent angiogenesis and tumor growth in experimental animals (Rozic J G et al., 2001, Int J Cancer, 93 :497-506; Liu XH et al., 2000, J. Urol, 164:820-5), but their efficacy for treatment of neoplasia and tumors as used in combination with thalidomide has not been demonstrated.

Angiogenesis is an attractive therapeutic target because it is a multi-step process that occurs in a specific sequence, thus providing several possible targets for drug action. Angiogenesis is important in two stages of tumor metastasis. The first stage where angiogenesis stimulation is important is in the vascularization of the tumor which allows the tumor cells to enter the blood stream and to circulate throughout the body. After the tumor cells have left the primary site and have settled into the secondary, metastasis site, angiogenesis must occur before the new tumor can grow and expand. Therefore, prevention of angiogenesis could lead to the prevention of metastasis of tumors and possibly contain the neoplastic growth at the primary site. Examples of agents that interfere with several of these steps include thrombospondin-1, angiostatin, endostatin, interferon alpha and compounds such as matrix metalloproteinase (MMP) inhibitors that block the actions of enzymes that clear and create paths for newly formed blood vessels to follow; compounds such as αᵥβ₃ inhibitors that interfere with molecules that blood vessel cells use to bridge between a patent blood vessel and a tumor; agents such as specific Cox-2 inhibitors that prevent the growth of cells that form new blood vessels; and protein-based compounds that simultaneously interfere with several of these targets.

Antiangiogenic therapy may offer several advantages over convential chemotherapy for the treatment of cancer, including the avoidance of the toxic side effects associated with chemotherapy and radiation.

Antiangiogenic agents have low toxicity in preclinical trials and development of drug resistance has not been observed (Folkman, J., Seminars in Medicine of the Beth Israel Hospital, Boston 333(26): 1757-1763, 1995). As angiogenesis is a complex process which is made up of many steps including invasion, proliferation and migration of endothelial cells, it can be anticipated that combinational therapies that target different steps of the process will be most effective. Kumar and Armstrong describe anti-angiogenesis therapy used as an adjunct to chemotherapy, radiation therapy or surgery (Kumar, C. C. and Armstrong, L., Tumor-induced Angiogenesis: A Novel Target for Drug Therapy?, Emerging Drugs, (1997) 2, 175-190).

Accordingly, the present inventive discovery is directed to the use of selective inhibitors of cyclooxygenase-2 in combination with thalidomide for the prevention or treatment of neoplasias such as cancer. More specifically, this inventive discovery relates to the use of cyclooxygenase-2 selective inhibitors or derivatives or pharmaceutically acceptable salts or prodrugs thereof in combination with thalidomide for preventing angiogenesis.

### SUMMARY OF THE INVENTION

Among the several aspects of the invention are provided compositions useful for treatment, prevention, or inhibition of neoplasia disorders, comprising a cyclooxygenase-2 (Cox-2) inhibitor or a pharmaceutically acceptable salt, ester or prodrug thereof in a first amount and a thalidomide, thalidomide analog, thalidomide hydrolysis product, thalidomide metabolite or thalidomide precursor in a second amount, wherein said first amount together with said second amount comprises a therapeutically effective amount for the treatment, prevention or inhibition of neoplasia disorders.

The present invention further provides methods for the treatment, prevention or inhibition of a neoplasia disorder in a subject in need of such treatment comprising administering to the subject a cyclooxygenase-2 inhibitor or a pharmaceutically acceptable salt, ester or prodrug thereof in a first amount and a thalidomide, thalidomide analog, thalidomide hydrolysis product, thalidomide metabolite or thalidomide precursor in a second amount, wherein said first amount together with said second amount comprises a therapeutically effective amount for the treatment, prevention or inhibition of neoplasia disorder in said subject.

Further provided are methods for treating, preventing or inhibiting angiogenesis by administering a cyclooxygenase-2 inhibitor or a pharmaceutically acceptable salt, ester or prodrug thereof in a first amount and a thalidomide, thalidomide analog, thalidomide hydrolysis product, thalidomide metabolite or thalidomide precursor in a second amount, wherein said first amount together with said second amount comprises a therapeutically effective amount for the treatment, prevention or inhibition of angiogenesis. Preferably, in the methods described herein, the subjects are animals, and more preferably the subjects are human. In one embodiment, Cox-2 inhibitors used in the methods and compositions described herein are selected from the group consisting of substituted benzothiopyrans, dihydroquinolines, or dihydronaphthalenes having the general Formula (I):

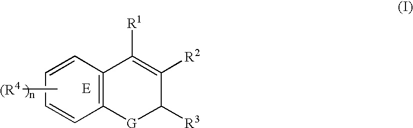

or an isomer, a pharmaceutically acceptable salt, an ester, or a prodrug thereof,

wherein n is an integer which is 0, 1, 2, 3 or 4;

wherein G is O, S or NRa;

wherein R^{a} is alkyl;

wherein R¹ is selected from the group consisting of H and aryl;

wherein R² is selected from the group consisting of carboxyl, aminocarbonyl, alkylsulfonylaminocarbonyl and alkoxycarbonyl;

wherein R³ is selected from the group consisting of haloalkyl, alkyl, aralkyl, cycloalkyl and aryl optionally substituted with one or more radicals selected from alkylthio, nitro and alkylsulfonyl; and

wherein each R⁴ is independently selected from the group consisting of one or more radicals selected from H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;

or wherein R⁴ together with carbon atoms to which it is attached and the remainder of the ring E forms a naphthyl radical;

or an isomer, a pharmaceutically acceptable salt, an ester, or a prodrug thereof,

In another embodiment, the Cox-2 inhibitors used herein have the general Formula (II):

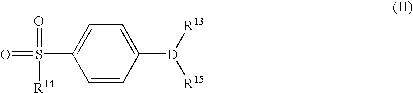

or an isomer, a pharmaceutically acceptable salt, an ester, or a prodrug thereof, wherein:

D is selected from the group consisting of partially unsaturated or saturated heterocyclyl and partially unsaturated or saturated carbocyclic rings;

R¹³ is selected from the group consisting of heterocyclyl, cycloalkyl, cycloalkenyl and aryl, wherein R¹³ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R¹⁴ is methyl or amino; and

R¹⁵ is H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, or N-alkyl-N-arylaminosulfonyl.

According to another embodiment, the present invention is also directed to novel compositions for the treatment, prevention or inhibition of neoplasia disorders comprising administering to a subject in need thereof, a cyclooxygenase-2 (Cox-2) inhibitor in a first amount and thalidomide in a second amount, wherein said first amount together with said second amount is a therapeutically effective amount of said Cox-2 inhibitor and thalidomide, and wherein said Cox-2 inhibitor comprises a phenylacetic acid derivative represented by the general Formula (III):

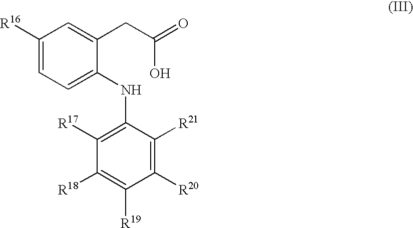

or an isomer, a pharmaceutically acceptable salt, an ester, or a prodrug thereof, wherein:

R¹⁶ is methyl or ethyl;

R¹⁷ is chloro or fluoro;

R¹⁸ is hydrogen or fluoro;

R¹⁹ is hydrogen, fluoro, chloro, methyl, ethyl, methoxy, ethoxy or hydroxy;

R²⁰ is hydrogen or fluoro; and

R²¹ is chloro, fluoro, trifluoromethyl or methyl, provided that R¹⁷, R¹⁸, R¹⁹ and R²⁰ are not all fluoro when R¹⁶ is ethyl and R¹⁹ is H.

In another embodiment, the Cox-2 inhibitors useful in the compositions and methods of the present invention are represented by Formula (IV):

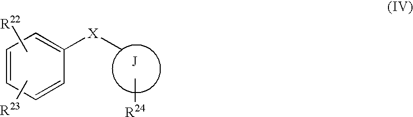

or an isomer, a pharmaceutically acceptable salt, an ester, or a prodrug thereof, wherein:

X is O or S;

J is a carbocycle or a heterocycle;

R²² is NHSO₂CH₃ or F;

R²³ is H, NO₂, or F; and

R²⁴ is H, NHSO₂CH₃, or (SO₂CH₃)C₆H₄.

According to another embodiment, the Cox-2 inhibitors described herein have structural Formula (V):

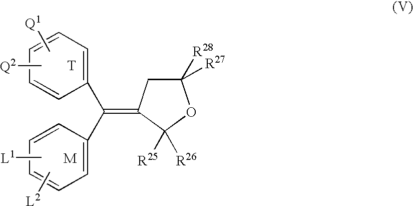

or an isomer, a pharmaceutically acceptable salt, an ester, or a prodrug thereof, wherein:

T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms; Q¹, Q², L¹ or L² are independently hydrogen, halogen, lower alkyl having from 1 to 6 carbon atoms, trifluoromethyl, or lower methoxy having from 1 to 6 carbon atoms; and

at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or,

Q¹ and Q² are methylenedioxy; or

L¹ and L² are methylenedioxy; and

R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,

R²⁵ and R²⁶ are O; or,

R²⁷ and R²⁸ are O; or,

R²⁵, R²⁶, together with the carbon atom to which they are attached, form a saturated hydrocarbon ring having from 3 to 7 carbon atoms; or,

R²⁷, R²⁸, together with the carbon atom to which they are attached, form a saturated hydrocarbon ring having from 3 to 7 carbon atoms.

In another embodiment, all of the Cox-2 inhibitors, in combination with a thalidomide, thalidomide analog, thalidomide hydrolysis product, thalidomide metabolite or thalidomide precursor may be used in the methods described herein for the treatment, prevention or inhibition of neoplasia disorders.

In another embodiment, a neoplasia disorder comprises a tumor growth. The tumor growth may be either malignant or benign.

Other aspects and features will be in part apparent and in part pointed out hereinafter.

### DETAILED DESCRIPTION OF THE INVENTION

The terms “hydrido” and “H” denote a single hydrogen atom. This hydrido radical may be attached, for example, to an oxygen atom to form a hydroxyl radical or two hydrido radicals may be attached to a carbon atom to form a methylene (—CH₂—) radical.

Where used, either alone or within other terms such as “haloalkyl”, “alkylsulfonyl”, “alkoxyalkyl” and “hydroxyalkyl”, the term “alkyl” embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like.

The term “alkenyl” embraces linear or branched radicals having at least one carbon-carbon double bond of two to about twenty carbon atoms or, preferably, two to about twelve carbon atoms. More preferred alkenyl radicals are “lower alkenyl” radicals having two to about six carbon atoms. Examples of alkenyl radicals include ethenyl, propenyl, allyl, propenyl, butenyl and 4-methylbutenyl.

The term “alkynyl” denotes linear or branched radicals having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like. The terms “alkenyl”, “lower alkenyl”, embrace radicals having “cis” and “trans” orientations, or alternatively, “E” and “Z” orientations.

The term “cycloalkyl” embraces saturated carbocyclic radicals having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl. The term “cycloalkenyl” embraces partially unsaturated carbocyclic radicals having three to twelve carbon atoms. More preferred cycloalkenyl radicals are “lower cycloalkenyl” radicals having four to about eight carbon atoms. Examples of such radicals include cyclobutenyl, cyclopentenyl, cyclopentadienyl and cyclohexenyl.

The term “halo” means halogens such as fluorine, chlorine, bromine or iodine. The term “haloalkyl” embraces radicals wherein any one or more of the alkyl carbon atoms is substituted with halo as defined above. Specifically embraced are monohaloalkyl, dihaloalkyl and polyhaloalkyl radicals. A monohaloalkyl radical, for one example, may have either an-iodo, bromo, chloro or fluoro atom within the radical. Dihalo and polyhaloalkyl radicals may have two or more of the same halo atoms or a combination of different halo radicals. “Lower haloalkyl” embraces radicals having one to six carbon atoms. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “hydroxyalkyl” embraces linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl.

The terms “alkoxy” and “alkyloxy” embrace linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy. The term “alkoxyalkyl” embraces alkyl radicals having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy.

The term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronaphthyl, indane and biphenyl. Aryl moieties may also be substituted at a substitutable position with one or more substituents selected independently from alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl.

The terms “heterocyclo”, “heterocyclyl”, and “heterocycle” embrace saturated, partially unsaturated and unsaturated heteroatom-containing ring-shaped radicals, where the heteroatoms may be selected from nitrogen, sulfur and oxygen. Examples of saturated heterocyclo radicals include saturated 3 to 6-membered heteromonocyclic groups containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclo radicals include dihydrothiophene, dihydropyran, dihydrofuiran and dihydrothiazole.

The term “heteroaryl” embraces unsaturated heterocyclo radicals. Examples of unsaturated heterocyclo radicals, also termed “heteroaryl” radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclo group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclo group containing 1 to 2 oxygen atoms and 1 to 3 nitrogen atoms (e.g. benzoxazolyl, benzoxadiazolyl, etc.); unsaturated 3 to 6-membered heteromonocyclic: group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclo group containing 1 to 2 sulfur atoms and 1 to 3 nitrogen atoms (e.g., benzothiazolyl, benzothiadiazolyl, etc.) and the like. The term also embraces radicals where heterocyclo radicals are fused with aryl radicals. Examples of such fused bicyclic radicals include benzofuran, benzothiophene, benzopyran, and the like. Said “heterocyclo group” may have 1 to 3 substituents such as alkyl, hydroxyl, halo, alkoxy, oxo, amino and alkylamino.

The term “alkylthio” embraces radicals containing a linear or branched alkyl radical, of one to about ten carbon atoms attached to a divalent sulfur atom. More preferred alkylthio radicals are “lower alkylthio” radicals having alkyl radicals of one to six carbon atoms. Examples of such lower alkylthio radicals are methylthio, ethylthio, propylthio, butylthio and hexylthio. The term “alkylthioalkyl” embraces radicals containing an alkylthio radical attached through the divalent sulfur atom to an alkyl radical of one to about ten carbon atoms. More preferred alkylthioalkyl radicals are “lower alkylthioalkyl” radicals having alkyl radicals of one to six carbon atoms. Examples of such lower alkylthioalkyl radicals include methylthiomethyl.

The term “alkylsulfinyl” embraces radicals containing a linear or branched alkyl radical, of one to ten carbon atoms, attached to a divalent —S(═O)— radical. More preferred alkylsulfinyl radicals are “lower alkylsulfinyl” radicals having alkyl radicals of one to six carbon atoms. Examples of such lower alkylsulfinyl radicals include methylsulfinyl, ethylsulfinyl, butylsulfinyl and hexylsulfinyl.

The term “sulfonyl”, whether used alone or linked to other terms such as alkylsulfonyl, denotes respectively divalent radicals —SO₂—. “Alkylsulfonyl” embraces alkyl radicals attached to a sulfonyl radical, where alkyl is defined as above. More preferred alkylsulfonyl radicals are “lower alkylsulfonyl” radicals having one to six carbon atoms. Examples of such lower alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals.

The terms “sulfamyl”, “aminosulfonyl” and “sulfonamidyl” denote NH₂O₂S—.

The term “acyl” denotes a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl and trifluoroacetyl.

The term “carbonyl”, whether used alone or with other terms, such as “alkoxycarbonyl”, denotes —(C═O)—. The term “aroyl” embraces aryl radicals with a carbonyl radical as defined above. Examples of aroyl include benzoyl, naphthoyl, and the like and the aryl in said aroyl may be additionally substituted.

The terms “carboxy” or “carboxyl”, whether used alone or with other terms, such as “carboxyalkyl”, denotes —CO₂H. The term “carboxyalkyl” embraces alkyl radicals substituted with a carboxy radical. More preferred are “lower carboxyalkyl” which embrace lower alkyl radicals as defined above, and may be additionally substituted on the alkyl radical with halo. Examples of such lower carboxyalkyl radicals include carboxymethyl, carboxyethyl and carboxypropyl.

The term “alkoxycarbonyl” means a radical containing an alkoxy radical, as defined above, attached via an oxygen atom to a carbonyl radical. More preferred are “lower alkoxycarbonyl” radicals with alkyl portions having 1 to 6 carbons. Examples of such lower alkoxycarbonyl (ester) radicals include substituted or unsubstituted methoxycarbonyl, ethoxycarbonyl, propoxycarbonyl, butoxycarbonyl and hexyloxycarbonyl.

The terms “alkylcarbonyl”, “arylcarbonyl” and “aralkylcarbonyl” include radicals having alkyl, aryl and aralkyl radicals, as defined above, attached to a carbonyl radical. Examples of such radicals include substituted or unsubstituted methylcarbonyl, ethylcarbonyl, phenylcarbonyl and benzylcarbonyl.

The term “aralkyl” embraces aryl-substituted alkyl radicals such as benzyl, diphenylmethyl, triphenylmethyl, phenylethyl, and diphenylethyl. The aryl in said aralkyl may be additionally substituted with halo, alkyl, alkoxy, haloalkyl and haloalkoxy. The terms benzyl and phenylmethyl are interchangeable.

The term “heterocycloalkyl” embraces saturated and partially unsaturated heterocyclo-substituted alkyl radicals, such as pyrrolidinylmethyl, and heteroarylsubstituted alkyl radicals, such as pyridylmethyl, quinolylmethyl, thienylmethyl, furylethyl, and quinolylethyl. The heteroaryl in said heteroaralkyl may be additionally substituted with halo, alkyl, alkoxy, haloalkyl and haloalkoxy.

The term “aralkoxy” embraces aralkyl radicals attached through an oxygen atom to other radicals. The term “aralkoxyalkyl” embraces aralkoxy radicals attached through an oxygen atom to an alkyl radical. The term “aralkylthio” embraces aralkyl radicals attached to a sulfur atom. The term “aralkylthioalkyl” embraces aralkylthio radicals attached through a sulfur atom to an alkyl radical.

The term “aminoalkyl” embraces alkyl radicals substituted with one or more amino radicals. More preferred are “lower aminoalkyl” radicals. Examples of such radicals include aminomethyl, aminoethyl, and the like. The term “alkylamino” denotes amino groups that have been substituted with one or two alkyl radicals. Preferred are “lower N-alkylamino” radicals having alkyl portions having 1 to 6 carbon atoms. Suitable lower alkylamino may be mono or dialkylamino such as N-methylamino, N-ethylamino, N,N-dimethylamino, N,N-diethylamino or the like. The term “arylamino” denotes amino groups that have been substituted with one or two aryl radicals, such as N-phenylamino. The “arylamino” radicals may be further substituted on the aryl ring portion of the radical. The term “aralkylamino” embraces aralkyl radicals attached through an amino nitrogen atom to other radicals. The terms “N-arylaminoalkyl” and “N-aryl-N-alkylaminoalkyl” denote amino groups which have been substituted with one aryl radical or one aryl and one alkyl radical, respectively, and having the amino group attached to an alkyl radical. Examples of such radicals include N-phenylaminomethyl and N-phenyl-N-methylaminomethyl.

The term “aminocarbonyl” denotes an amide group of the formula —C(═O)NH₂. The term “alkylaminocarbonyl” denotes an aminocarbonyl group that has been substituted with one or two alkyl radicals on the amino nitrogen atom. Preferred are “N-alkylaminocarbonyl” and “N,N-dialkylaminocarbonyl” radicals. More preferred are “lower N-alkylaminocarbonyl” and “lower N,N-dialkylaminocarbonyl” radicals with lower alkyl portions as defined above. The term “aminocarbonylalkyl” denotes a carbonylalkyl group that has been substituted with an amino radical on the carbonyl carbon atom.

The term “alkylaminoalkyl” embraces radicals having one or more alkyl radicals attached to an aminoalkyl radical. The term “aryloxyalkyl” embraces radicals having an aryl radical attached to an alkyl radical through a divalent oxygen atom. The term “arylthioalkyl” embraces radicals having an aryl radical attached to an alkyl radical through a divalent sulfur atom.

As used herein, the term “carbocycle” means a hydrocarbon ring radical. Carbocyclic rings are monocyclic or are fused, bridged, or spiro polycyclic rings. Unless otherwise specified, monocyclic rings contain from 3 to about 9 atoms, preferably from about 4 to about 7 atoms, and most preferably 5 or 6 atoms. Polycyclic rings contain from about 7 to about 17 atoms, preferably from about 7 to about 14 atoms, and most preferably 9 or 10 atoms. Carbocyclic rings (carbocycles) may be substituted or unsubstituted.

The symbol “Me” means methyl or CH₃.

The symbol “Et” means ethyl or CH₃CH₂.

The symbol “Ac” means acetyl or COCH₃.

The symbol “OAc” means OCOCH₃.

As used herein, the term “neoplasia” refers to any new or abnormal growth of cells, as well as to diseases related to neoplasia. Accordingly, diseases which may be treated by the methods, compounds and compositions of this invention, include, but are not limited to, tumor growth or tumor cell growth, including benign tumor growth and malignant tumor growth, metastasis, acral lentiginous melanoma, actinic keratoses, adenocarcinoma, adenoid cystic carcinoma, adenomas, adenosarcoma, adenosquamous carcinoma, astrocytic tumors, bartholin gland carcinoma, basal cell carcinoma, blastoma, breast cancer including benign tumor growth in the breast, bronchial gland carcinomas, capillary, carcinoids, carcinoma, carcinosarcoma, cavernous, cholangiocarcinoma, chondrosarcoma, choriod plexus papilloma/carcinoma, clear cell carcinoma, cystadenoma, cyst, ovarian cyst, endodermal sinus tumor, endometrial hyperplasia, endometrial stromal sarcoma, endometrioid adenocarcinoma, ependymal, epitheloid, Ewing's sarcoma, fibrolamellar, fibroma, fibroid tumor, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intaepithelial neoplasia, interepithelial squamous cell neoplasia, invasive squamous cell carcinoma, large cell carcinoma, leiomyosarcoma, lentigo maligna melanomas, lipoma, malignant melanoma, malignant mesothelial tumors, medulloblastoma, medulloepithelioma, melanoma, meningeal, mesothelial, metastatic carcinoma, mucoepidermoid carcinoma, myoma, neuroblastoma, neuroepithelial adenocarcinoma nodular melanoma, oat cell carcinoma, oligodendroglial, osteosarcoma, pancreatic polypeptide, papillary serous adenocarcinoma, pineal cell, pituitary tumors, polyp, plasmacytoma, pseudosarcoma, pulmonary blastoma, renal cell carcinoma, retinoblastoma, rhabdomyosarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiatied carcinoma, uveal melanoma, verrucous carcinoma, vipoma, well differentiated carcinoma, and Wilm's tumor.

In one embodiment of the present invention, malignant growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis. Malignant growth can also include viral-related cancers, including but not restricted to cervical cancer, T-cell leukemia, lymphoma, and Kaposi's sarcoma.

In another embodiment, the benign tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis. In another embodiment, the benign tumor growth is a fibroid tumor, an endometriosis, or a cyst.

As used herein, the term “purified” means partially purified and/or completely purified. Thus a “purified composition” may be either partially purified or completely purified. The Cox-2 inhibitor(s), as well as thalidomide, useful in the inventive method for treating neoplasia disorders, can be of any purity and quality such that the combination for the Cox-2 inhibitor(s) and thalidomide is pharmaceutically acceptable.

As used herein, the term “thalidomide” includes analogs, hydrolysis products, metabolites, and precursors thereof unless the context precludes it. Thalidomide analogs, hydrolysis products, metabolites, or precursors, and methods of synthesizing such compounds are disclosed in PCT/US97/20116 and U.S. Pat. No. 6,235,756.

The present disclosure provides a method for treating, preventing or inhibiting neoplasia disorders in a subject in need of such treatment, prevention or inhibition. The method comprises administering to the subject a therapeutically effective amount of a cyclooxygenase-2 selective inhibitor or prodrug, ester or pharmaceutically acceptable salt thereof in combination with thalidomide.

Without being limited to any particular theory, the administration of an effective amount of thalidomide, a thalidomide analog, a thalidomide hydrolysis product, a thalidomide metabolite, or a precursor of thalidomide preferably inhibits endothelial cell proliferation and tube formation, having the effect of preventing new capillary blood vessels from forming and thus inhibiting angiogenesis. The ingrowth of capillaries and ancillary blood vessels is essential for growth of solid tumors and is thus an unwanted physiological response which facilitates the spread of malignant tissue and metastases. Inhibition of angio genesis and the resultant growth of capillaries and blood vessels is therefore a component of effective treatment of malignancy.

Inhibitors of the cyclooxygenase pathway in the metabolism of arachidonic acid used in the present method may inhibit enzyme activity through a variety of mechanisms. By way of example, and without limitation, the inhibitors used in the methods described herein potentiate tumor response to treatment with thalidomide. The use of cyclooxygenase-2 selective inhibitors is highly advantageous in the present methods in that they improve the efficacy of thalidomide in the inhibition of angiogenesis and thus, reduce cancerous tumor growth.

In an embodiment of the present invention, any cyclooxygenase-2 selective inhibitor or isomer, pharmaceutically acceptable salt, ester, or prodrugs thereof that meets the criteria described below can be used, along with thalidomide, thalidomide analog, thalidomide hydrolysis product, thalidomide metabolite or thalidomide precursor as described below, in the subject inventive method.

As used herein, the term “cyclooxygenase-2 inhibitor”, embraces compounds which selectively inhibit cyclooxygenase-2 over cyclooxygenase-1, and also includes pharmaceutically acceptable salts of those compounds.

In practice, the selectivity of a Cox-2 inhibitor varies depending upon the condition under which the test is performed and on the inhibitors being tested. However, for the purposes of this specification, the selectivity of a Cox-2 inhibitor can be measured as a ratio of the in vitro or in vivo IC₅₀ value for inhibition of Cox-1, divided by the IC₅₀ value for inhibition of Cox-2 (Cox-1 IC₅₀/Cox-2 IC₅₀). A Cox-2 selective inhibitor is any inhibitor for which the ratio of Cox-1 IC₅₀ to Cox-2 IC₅₀ is greater than 1, preferably greater than 1.5, more preferably greater than 2, even more preferably greater than 5, yet more preferably greater than 10, still more preferably greater than 50, and more preferably still greater than 100.

As used herein, the term “IC₅₀” refers to the concentration of a compound that is required to produce 50% inhibition of cyclooxygenase activity. Preferred cyclooxygenase-2 selective inhibitors of the present invention have a cyclooxygenase-2 IC₅₀ of less than about 5 μM, more preferred of less than about 1 μM.

Preferred cycloxoygenase-2 selective inhibitors have a cyclooxygenase-1 IC₅₀ of greater than about 1 μM, and more preferably of greater than 20 μM. Such preferred selectivity may indicate an ability to reduce the incidence of common NSAID-induced side effects.

In another embodiment, a preferred Cox-2 inhibitor or isomer, pharmaceutically acceptable salt, ester, or prodrug thereof has a selectivity ratio of Cox-2 inhibition to Cox-1 inhibition of at least about 1.5, and more preferably of at least about 100.

Also included within the scope of the present invention are compounds that act as prodrugs of cyclooxygenase-2-selective inhibitors. As used herein in reference to Cox-2 selective inhibitors, the term “prodrug” refers to a chemical compound that is converted into an active Cox-2 selective inhibitor by metabolic processes within the body. One example of a prodrug for a Cox-2 selective inhibitor is parecoxib, which is a therapeutically effective prodrug of the tricyclic cyclooxygenase-2 selective inhibitor valdecoxib. An example of a preferred Cox-2 selective inhibitor prodrug is sodium parecoxib.

The cyclooxygenase-2 selective inhibitor of the present invention can be, for example, the Cox-2 selective inhibitor [2-(2,4-Dichloro-6-ethyl-3,5-dimethyl-phenylamino)-5-propyl-phenyl]-acetic acid, having Formula B-1, or an isomer or pharmaceutically acceptable salt, ester, or prodrug thereof.

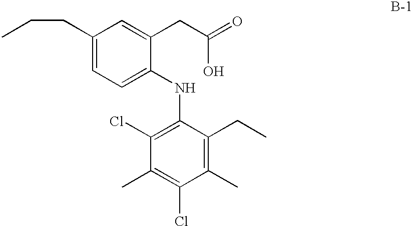

In another embodiment of the invention the cyclooxygenase-2 selective inhibitor can be the Cox-2 selective inhibitor RS 57067 or 6-[5-(4-chlorobenzoyl)-1,4-dimethyl-1H-pyrrol-2-yl]methyl-3(2H)-pyridazinone, having Formula B-2 (CAS registry number 179382-91-3), or an isomer, a pharmaceutically acceptable salt, or prodrug thereof.

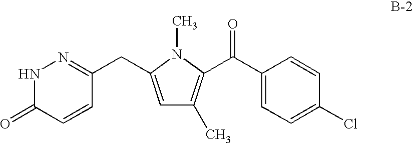

In a preferred embodiment of the invention the cyclooxygenase-2 selective inhibitor is of the chromene structural class that is a substituted benzopyran or a substituted benzopyran analog, and even more preferably selected from the group consisting of substituted benzothiopyrans, dihydroquinolines, or dihydronaphthalenes having a structure shown by general Formula I, shown below, and possessing, by way of example and not limitation, the structures disclosed in Table 1, including the diastereomers, enantiomers, racemates, tautomers, salts, esters, amides and prodrugs thereof.

Furthermore, benzopyran Cox-2 selective inhibitors useful in the practice of the present invention are described in U.S. Pat. Nos. 6,034,256 and 6,077,850. Formula (I) is:

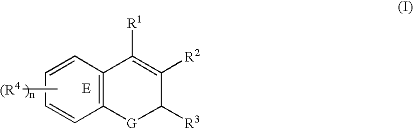

or an isomer, a pharmaceutically acceptable salt, ester, or prodrug thereof,

wherein n is an integer which is 0, 1, 2, 3 or 4;

wherein G is O, S or NR^{a};

wherein R^{a} is alkyl;

wherein R¹ is selected from the group consisting of H and aryl;

wherein R² is selected from the group consisting of carboxyl, aminocarbonyl, alkylsulfonylaminocarbonyl and alkoxycarbonyl;

wherein R³ is selected from the group consisting of haloalkyl, alkyl, aralkyl, cycloalkyl and aryl optionally substituted with one or more radicals selected from alkylthio, nitro and alkylsulfonyl; and

wherein each R⁴ is independently selected from the group consisting of H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl;

or wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof; wherein:

n is an integer which is 0, 1, 2, 3 or 4; wherein:

G is O, S or NR^{b};

R¹ is H;

R^{b} is alkyl;

R² is selected from the group consisting of carboxyl, aminocarbonyl, alkylsulfonylaminocarbonyl and alkoxycarbonyl;

R³ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and

each R⁴ is independently selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁴ together with ring E forms a naphthyl radical.

The cyclooxygenase-2 selective inhibitor may also be a compound of Formula (I), or an isomer, a pharmaceutically acceptable salt, ester, or prodrug thereof; wherein:

n is an integer which is 0, 1, 2, 3 or 4;

G is oxygen or sulfur;

R¹ is H;

R² is carboxyl, lower alkyl, lower aralkyl or lower alkoxycarbonyl;

R³ is lower haloalkyl, lower cycloalkyl or phenyl; and

each R⁴ is H, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, or lower alkylcarbonyl; or

wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof; wherein:

R² is carboxyl;

R³ is lower haloalkyl; and

each R⁴ is H, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, or lower alkylcarbonyl; or wherein R⁴ together with ring E forms a naphthyl radical.

The cyclooxygenase-2 selective inhibitor may also be a compound of Formula (I) or an isomer, a pharmaceutically acceptable salt, ester, or prodrug thereof, wherein:

n is an integer which is 0, 1, 2, 3 or 4;

R³ is fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, or trifluoromethyl; and

each R⁴ is H, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl or phenyl; or wherein R⁴ together with the carbon atoms to which it is attached and the remainder of ring E forms a naphthyl radical.

The cyclooxygenase-2 selective inhibitor may also be a compound of Formula (I) or an isomer, a pharmaceutically acceptable salt, ester, or prodrug thereof; wherein:

n is an integer which is 0, 1, 2, 3 or 4;

R³ is trifluoromethyl or pentafluoroethyl; and

each R⁴ is independently H, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, or phenyl; or wherein R⁴ together with the carbon atoms to which it is attached and the remainder of ring E forms a naphthyl radical.

The cyclooxygenase-2 selective inhibitor used in connection with the method(s) of the present invention can also be a compound having the structure of Formula (I) or an isomer, a pharmaceutically acceptable salt, ester, or prodrug thereof: wherein:

n=4;

G is O or S;

R¹ is H;

R² is CO₂H;

R³ is lower haloalkyl;

a first R⁴ corresponding to R⁹ is hydrido or halo;

a second R⁴ corresponding to R¹⁰ is H, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, or 6-membered nitrogen-containing heterocyclosulfonyl;

a third R⁴ corresponding to R¹¹ is H, lower alkyl, halo, lower alkoxy, or aryl; and

a fourth R⁴ corresponding to R¹² is H, halo, lower alkyl, lower alkoxy, and aryl;

wherein Formula (I) is represented by Formula (Ia):

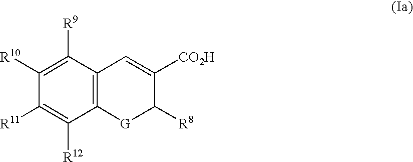

or an isomer, a pharmaceutically acceptable salt, ester, or prodrug thereof.

The cyclooxygenase-2 selective inhibitor used in connection with the method(s) of the present invention can also be a compound of having the structure of Formula (Ia) or an isomer, a pharmaceutically acceptable salt, ester, or prodrug thereof; wherein:

R⁸ is trifluoromethyl or pentafluoroethyl;

R⁹ is H, chloro, or fluoro;

R¹⁰ is H, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, or morpholinosulfonyl;

R¹¹ is H, methyl, ethyl, isopropyl, tert-butyl, chloro, methoxy, diethylamino, or phenyl; and

R¹² is H, chloro, bromo, fluoro, methyl, ethyl, tert-butyl, methoxy, or phenyl.

The present invention is also directed to a novel method for the treatment of neoplasia disorders comprising administering to a subject in need thereof a therapeutically effective amount of a cyclooxygenase-2 selective inhibitor comprising BMS-347070 (B-74), ABT 963 (B-25), NS-398 (B-26), L-745337 (B-214), RWJ-63556 (B-215), or L-784512 (B-216).

Of the Cox-2 inhibitors listed in Table 1A, those listed in Table 1B are chromene Cox-2 inhibitors as indicated below:

TABLE 1B
Examples of Chromene Cox-2 Selective Inhibitors

| No. | Structure (chromene Cox-2 Inhibitor) |
|---|---|
| B-3 | 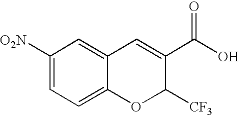 |
| | 6-Nitro-2-trifluoromethyl-2H-1- |
| | benzopyran-3-carboxylic acid |
| B-4 | 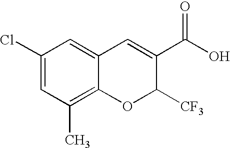 |
| | 6-Chloro-8-methyl-2-trifluoromethyl- |
| | 2H-1-benzopyran-3-carboxylic acid |
| B-5 | 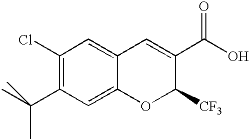 |
| | ((S)-6-Chloro-7-(1,1-dimethylethyl)-2-(trifluoromethyl- |
| | 2H-1-benzopyran-3-carboxylic acid |
| B-6 | 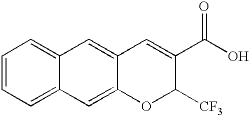 |
| | 2-Trifluoromethyl-2H-naphtho[2,3-b] |
| | pyran-3-carboxylic acid |
| B-7 | 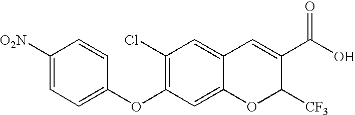 |
| | 6-Chloro-7-(4-nitrophenoxy)-2-(trifluoromethyl)-2H-1- |
| | benzopyran-3-carboxylic acid |
| B-8 | 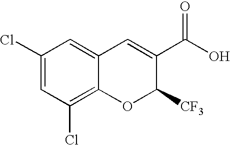 |
| | ((S)-6,8-Dichloro-2-(trifluoromethyl)- |
| | 2H-1-benzopyran-3-carboxylic acid |
| B-9 | 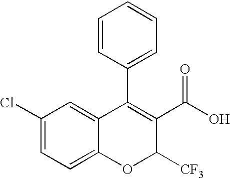 |
| | 6-Chloro-2-(trifluoromethyl)-4-phenyl-2H- |
| | 1-benzopyran-3-carboxylic acid |
| B-10 | 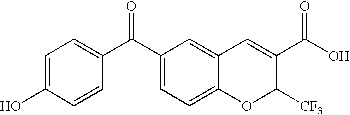 |
| | 6-(4-Hydroxybenzoyl)-2-(trifluoromethyl)- |
| | 2H-1-benzopyran-3-carboxylic acid |
| B-11 | 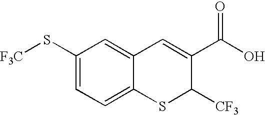 |
| | 2-(Trifluoromethyl)-6-[(trifluoromethyl)thio]- |
| | 2H-1-benzothiopyran-3-carboxylic acid |
| B-12 | 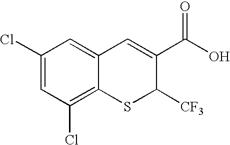 |
| | 6,8-Dichloro-2-trifluoromethyl-2H-1- |
| | benzothiopyran-3-carboxylic acid |
| B-13 | 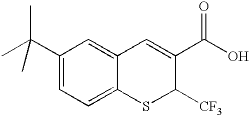 |
| | 6-(1,1-Dimethylethyl)-2-(trifluoromethyl)- |
| | 2H-1-benzothiopyran-3-carboxylic acid |
| B-14 | 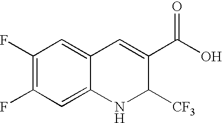 |
| | 6,7-Difluoro-1,2-dihydro-2-(trifluoromethyl)- |
| | 3-quinolinecarboxylic acid |
| B-15 | 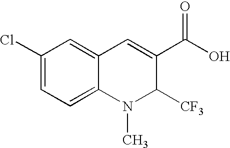 |
| | 6-Chloro-1,2-dihydro-1-methyl-2-(trifluoromethyl)- |
| | 3-quinolinecarboxylic acid |
| B-16 | 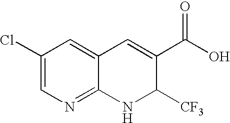 |
| | 6-Chloro-2-(trifluoromethyl)-1,2-dihydro |
| | [1,8]naphthyridine-3-carboxylic acid |
| B-17 | 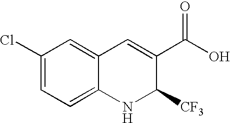 |
| | ((S)-6-Chloro-1,2-dihydro-2-(trifluoromethyl)- |
| | 3-quinolinecarboxylic acid |

In a further preferred embodiment of the invention the cyclooxygenase inhibitor, when used in combination with thalidomide can be selected from the class of tricyclic cyclooxygenase-2 selective inhibitors represented by the general structure of Formula (II):

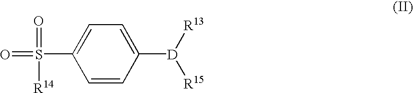

or an isomer, a pharmaceutically acceptable salt, ester, or prodrug thereof, wherein:

D is selected from the group consisting of partially unsaturated or unsaturated heterocyclyl and partially unsaturated or unsaturated carbocyclic rings;

R¹³ is selected from the group consisting of heterocyclyl, cycloalkyl, cycloalkenyl and aryl, wherein R¹³ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R¹⁴ is selected from the group consisting of methyl or amino; and

R¹⁵ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N- arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl.

In a still more preferred embodiment of the invention, the tricyclic cyclooxygenase-2 selective inhibitor(s), for use in connection with the method(s) of the present invention and in combination with thalidomide are represented by the above Formula (II) and are selected from the group of compounds, illustrated in Table 2, consisting of celecoxib (B-18), valdecoxib (B-19), deracoxib (B-20), rofecoxib (B-21), etoricoxib (MK-663; B-22), JTE-522 (B-23), or an isomer, a pharmaceutically acceptable salt, ester, or prodrug thereof.

TABLE 2
Examples of Tricyclic COX-2 Selective Inhibitors

| No. | Structure (Tricyclic Cox-2 Inhibitors) |
|---|---|
| B-18 | 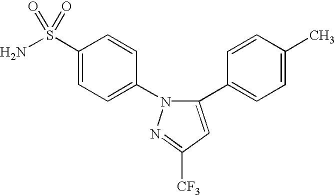 |
| | celecoxib |
| B-19 | 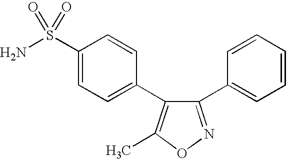 |
| | valdecoxib |
| B-20 | 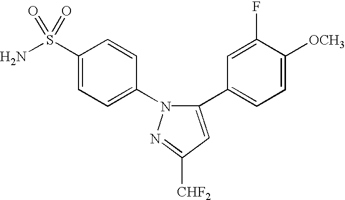 |
| | deracoxib |
| B-21 | 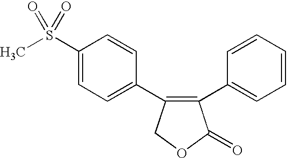 |
| | rofecoxib |
| B-22 | 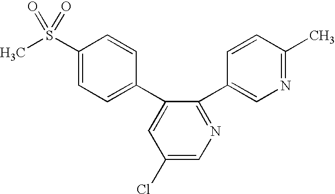 |
| | etoricoxib |
| B-23 | 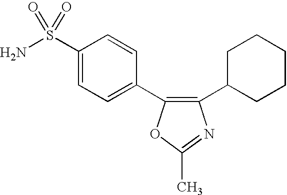 |
| | JTE-522 |

In an even more preferred embodiment of the invention, the Cox-2 selective inhibitor, when used in combination with thalidomide is selected from the group consisting of celecoxib, rofecoxib and etoricoxib.

In another preferred embodiment of the invention, parecoxib, (B-24), which is a therapeutically effective prodrug of the tricyclic cyclooxygenase-2 selective inhibitor valdecoxib, (B-19), may be advantageously employed as a source of a cyclooxygenase inhibitor (See, e.g., U.S. Pat. No. 5,932,598) in connection with the method(s) in the present invention.

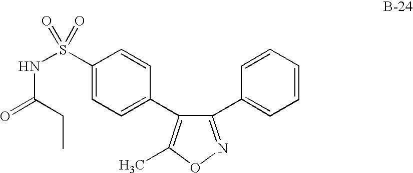

A preferred form of parecoxib is sodium parecoxib.

In another preferred embodiment of the invention, the compound ABT-963 having the formula (B-25) that has been previously described in International Publication number WO 00/24719, is another tricyclic cyclooxygenase-2 selective inhibitor which may be advantageously employed in connection with the method(s) of the present invention.

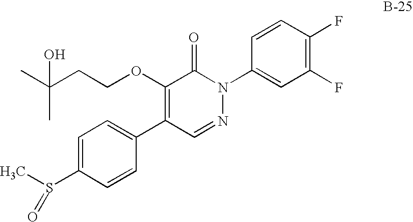

Another preferred cyclooxygenase-2 selective inhibitor that is useful in connection with the method(s) of the present invention is N-(2-cyclohexyloxynitrophenyl)-methane sulfonamide (NS-398)—having a structure shown below as B-26. Applications of this compound have been described by, for example, Yoshimi, N. et al., in Japanese J. Cancer Res., 90(4):406-412 (1999); Falgueyret, J. -P. et al., in Science Spectra, available at: http://www.gbhap.com/Science_Spectra/20-1-article.htm (Jun. 6, 2001); and Iwata, K. et al., in Jpn. J. Pharmacol., 75(2):191-194 (1997).

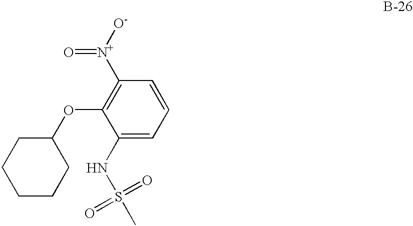

Other compounds that are useful for the cyclooxygenase-2 selective inhibitor in connection with the method(s) of the present invention include, but are not limited to:

6-chloro-2-trifluoromethyl-2H-1-benzopyran-3-carboxylic acid (B-27);

6-chloro-7-methyl-2-trifluoromethyl-2H-1-benzopyran-3-carboxylic acid (B-28);

8-(1-methylethyl)-2-trifluoromethyl-2H-1-benzopyran-3-carboxylic acid (B-29);

6-chloro-8-(1-methylethyl)-2-trifluoromethyl-2H-1-benzopyran-3-carboxylic acid (B-30);

2-trifluoromethyl-3H-naphtho[2,1-b]pyran-3-carboxylic acid (B-31);

7-(1,1-dimethylethyl)-2-trifluoromethyl-2H- 1 -benzopyran-3-carboxylic acid (B-32);

6-bromo-2-trifluoromethyl-2H-1-benzopyran-3-carboxylic acid (B-33);

8-chloro-2-trifluoromethyl-2H-1-benzopyran-3-carboxylic acid (B-34);

6-trifluoromethoxy-2-trifluoromethyl-2H-1-benzopyran-3-carboxylic acid (B-35);

5,7-dichloro-2-trifluoromethyl-2H-1-benzopyran-3-carboxylic acid (B-36);

8-phenyl-2-trifluoromethyl-2H-1-benzopyran-3-carboxylic acid (B-37);

7,8-dimethyl-2-trifluoromethyl-2H-1-benzopyran-3-carboxylic acid (B-38);

6,8-bis(dimethylethyl)-2-trifluoromethyl-2H-1-benzopyran-3-carboxylic acid (B-39);

7-(1-methylethyl)-2-trifluoromethyl-2H-1-benzopyran-3-carboxylic acid (B-40);

7-phenyl-2-trifluoromethyl-2H-1-benzopyran-3-carboxylic acid (B-41);

6-chloro-7-ethyl-2-trifluoromethyl-2H-1-benzopyran-3-carboxylic acid (B-42);

6-chloro-8-ethyl-2-trifluoromethyl-2H-1-benzopyran-3-carboxylic acid (B-43);

6-chloro-7-phenyl-2-trifluoromethyl-2H-1-benzopyran-3-carboxylic acid (B-44);

6,7-dichloro-2-trifluoromethyl-2H-1-benzopyran-3-carboxylic acid (B-45);

6,8-dichloro-2-trifluoromethyl-2H-1-benzopyran-3-carboxylic acid (B-46);

6-chloro-8-methyl-2-trifluoromethyl-2H-1-benzopyran-3-carboxylic acid (B-47);

8-chloro-6-methyl-2-trifluoromethyl-2H-1-benzopyran-3-carboxylic acid (B-48)

8-chloro-6-methoxy-2-trifluoromethyl-2H-1-benzopyran-3-carboxylic acid (B-49);

6-bromo-8-chloro-2-trifluoromethyl-2H-1-benzopyran-3-carboxylic acid (B-50);

8-bromo-6-fluoro-2-trifluoromethyl-2H-1-benzopyran-3-carboxylic acid (B-51);

8-bromo-6-methyl-2-trifluoromethyl-2H-1-benzopyran-3-carboxylic acid (B-52);

8-bromo-5-fluoro-2-trifluoromethyl-2H-1-benzopyran-3-carboxylic acid (B-53);

6-chloro-8-fluoro-2-trifluoromethyl-2H-1-benzopyran-3-carboxylic acid (B-54);

6-bromo-8-methoxy-2-trifluoromethyl-2H-1-benzopyran-3-carboxylic acid (B-55);

6-[[(phenylmethyl)amino]sulfonyl]-2-trifluoromethyl-2H-1-benzopyran-3-carboxylic acid (B-56);

6-[(dimethylamino)sulfonyl]-2-trifluoromethyl-2H-1-benzopyran-3-carboxylic acid (B-57);

6-[(methylamino)sulfonyl]-2-trifluoromethyl-2H-1-benzopyran-3-carboxylic acid (B-58);

6-[(4-morpholino)sulfonyl]-2-trifluoromethyl-2H-1-benzopyran-3-carboxylic acid (B-59);

6-[(1,1-dimethylethyl)aminosulfonyl]-2-trifluoromethyl-2H-1-benzopyran-3-carboxylic acid (B-60);

6-[(2-methylpropyl)aminosulfonyl]-2-trifluoromethyl-2H-1-benzopyran-3-carboxylic acid (B-61);

6-methylsulfonyl-2-trifluoromethyl-2H-1-benzopyran-3-carboxylic acid (B-62);

8-chloro-6-[[(phenylmethyl)amino]sulfonyl]-2-trifluoromethyl-2H-1-benzopyran-3-carboxylic acid (B-63);

6-phenylacetyl-2-trifluoromethyl-2H-1-benzopyran-3-carboxylic acid (B-64);

6,8-dibromo-2-trifluoromethyl-2H-1-benzopyran-3-carboxylic acid (B-65);

8-chloro-5,6-dimethyl-2-trifluoromethyl-2H-1-benzopyran-3-carboxylic acid (B-66);

6,8-dichloro-(S)-2-trifluoromethyl-2H-1-benzopyran-3-carboxylic acid (B-67);

6-benzylsulfonyl-2-trifluoromethyl-2H-1-benzopyran-3-carboxylic acid (B-68);

6-[[N-(2-furylmethyl)amino]sulfonyl]-2-trifluoromethyl-2H-1-benzopyran-3-carboxylic acid (B-69);

6-[[N-(2-phenylethyl)amino]sulfonyl]-2-trifluoromethyl-2H-1-benzopyran-3-carboxylic acid (B-70);

6-iodo-2-trifluoromethyl-2H-1-benzopyran-3-carboxylic acid (B-71);

7-(1,1-dimethylethyl)-2-pentafluoroethyl-2H-1-benzopyran-3-carboxylic acid (B-72);

6-chloro-2-trifluoromethyl-2H-1-benzothiopyran-3-carboxylic acid (B-73);

3-[(3-Chloro-phenyl)-(4-methanesulfonyl-phenyl)-methylene]-dihydro-furan-2-one or BMS-347070 (B-74);

8-acetyl-3-(4-fluorophenyl)-2-(4-methylsulfonyl)phenyl-imidazo(1,2-a)pyridine (B-75);

5,5-dimethyl-4-(4-methylsulfonyl)phenyl-3-phenyl-2-(5H)-furanone (B-76);

5-(4-fluorophenyl)-1-[4-(methylsulfonyl)phenyl]-3-(trifluoromethyl)pyrazole (B-77);

4-(4-fluorophenyl)-5-[4-(methylsulfonyl)phenyl]-1-phenyl-3-(trifluoromethyl)pyrazole (B-78);

4-(5-(4-chlorophenyl)-3-(4-methoxyphenyl)-1H-pyrazol-1-yl)benzenesulfonamide (B-79);

4-(3,5-bis(4-methylphenyl)-1H-pyrazol-1-yl)benzenesulfonamide (B-80);

4-(5-(4-chlorophenyl)-3-phenyl-1H-pyrazol-1-yl)benzenesulfonamide (B-81);

4-(3,5-bis(4-methoxyphenyl)-1H-pyrazol-1-yl)benzenesulfonamide (B-82);

4-(5-(4-chlorophenyl)-3-(4-methylphenyl)-1H-pyrazol-1-yl)benzenesulfonamide (B-83);

4-(5-(4-chlorophenyl)-3-(4-nitrophenyl)-1H-pyrazol-1-yl)benzenesulfonamide (B-84);

4-(5-(4-chlorophenyl)-3-(5-chloro-2-thienyl)-1H-pyrazol-1-yl)benzenesulfonamide (B-85);

4-(4-chloro-3,5-diphenyl-1H-pyrazol-1-yl)benzenesulfonamide (B-86);

4-[5-(4-chlorophenyl)-3-(trifluoromethyl)-1H-pyrazol-1-yl]benzenesulfonamide (B-87);

4-[5-phenyl-3-(trifluoromethyl)-1H-pyrazol-1-yl]benzenesulfonamide (B-88);

4-[5-(4-fluorophenyl)-3-(trifluoromethyl)-1H-pyrazol-1-yl]benzenesulfonamide (B-89);

4-[5-(4-methoxyphenyl)-3-(trifluoromethyl)-1H-pyrazol-1-yl]benzenesulfonamide (B-90);

4-[5-(4-chlorophenyl)-3-(difluoromethyl)-1H-pyrazol-1-yl]benzenesulfonamide (B-91);

4-[5-(4-methylphenyl)-3-(trifluoromethyl)-1H-pyrazol-1-yl]benzenesulfonamide (B-92);

4-[4-chloro-5-(4-chlorophenyl)-3-(trifluoromethyl)-1H-pyrazol-1-yl]benzenesulfonamide (B-93);

4-[3-(difluoromethyl)-5-(4-methylphenyl)-1H-pyrazol-1-yl]benzenesulfonamide (B-94);

4-[3-(difluoromethyl)-5-phenyl-1H-pyrazol-1-yl]benzenesulfonamide (B-95);

4-[3-(difluoromethyl)-5-(4-methoxyphenyl)-1H-pyrazol-1-yl]benzenesulfonamide (B-96);

4-[3-cyano-5-(4-fluorophenyl)-1H-pyrazol-1-yl]benzenesulfonamide (B-97);

4-[3-(difluoromethyl)-5-(3-fluoro-4-methoxyphenyl)-1H-pyrazol-1-yl]benzenesulfonamide (B-98);

4-[5-(3-fluoro-4-methoxyphenyl)-3-(trifluoromethyl)-1H-pyrazol-1-yl]benzenesulfonamide (B-99);

4-[4-chloro-5-phenyl-1H-pyrazol-1-yl]benzenesulfonamide (B-100);

4-[5-(4-chlorophenyl)-3-(hydroxymethyl)-1H-pyrazol-1-yl]benzenesulfonamide (B-101);

4-[5-(4-(N,N-dimethylamino)phenyl)-3-(trifluoromethyl)-1H-pyrazol-1-yl]benzenesulfonamide (B-102);

5-(4-fluorophenyl)-6-[4-(methylsulfonyl)phenyl]spiro[2.4]hept-5-ene (B-103);

4-[6-(4-fluorophenyl)spiro[2.4]hept-5-en-5-yl]benzenesulfonamide (B-104);

6-(4-fluorophenyl)-7-[4-(methylsulfonyl)phenyl]spiro[3.4]oct-6-ene (B-105);

5-(3-chloro-4-methoxyphenyl)-6-[4-(methylsulfonyl)phenyl]spiro[2.4]hept-5-ene (B-106);

4-[6-(3-chloro-4-methoxyphenyl)spiro[2.4]hept-5-en-5-yl]benzenesulfonamide (B-107);

5-(3,5-dichloro-4-methoxyphenyl)-6-[4-(methylsulfonyl)phenyl]spiro[2.4]hept-5-ene (B-108);

5-(3-chloro-4-fluorophenyl)-6-[4-(methylsulfonyl)phenyl]spiro[2.4]hept-5-ene (B-109);

4-[6-(3,4-dichlorophenyl)spiro[2.4]hept-5-en-5-yl]benzenesulfonamide (B-110);

2-(3 -chloro-4-fluorophenyl)-4-(4-fluorophenyl)-5 -(4-methylsulfonylphenyl)thiazole (B-111);

2-(2-chlorophenyl)-4-(4-fluorophenyl)-5 -(4-methylsulfonylphenyl)thiazole (B-112);

5-(4-fluorophenyl)-4-(4-methylsulfonylphenyl)-2-methylthiazole (B-113);

4-(4-fluorophenyl)-5-(4-methylsulfonylphenyl)-2-trifluoromethylthiazole (B-114);

4-(4-fluorophenyl)-5-(4-methylsulfonylphenyl)-2-(2-thienyl)thiazole (B-115);

4-(4-fluorophenyl)-5-(4-methylsulfonylphenyl)-2-benzylaminothiazole (B-116);

4-(4-fluorophenyl)-5-(4-methylsulfonylphenyl)-2-(1-propylamino)thiazole (B-117);

2-[(3,5-dichlorophenoxy)methyl)-4-(4-fluorophenyl)-5-[4-(methylsulfonyl)phenyl]thiazole (B-118);

5-(4-fluorophenyl)-4-(4-methylsulfonylphenyl)-2-trifluoromethylthiazole (B-119);

1-methylsulfonyl-4-[1,1 -dimethyl-4-(4-fluorophenyl)cyclopenta-2,4-dien-3-yl]benzene (B-120);

4-[4-(4-fluorophenyl)-1,1-dimethylcyclopenta-2,4-dien-3-yl]benzenesulfonamide (B-121);

5-(4-fluorophenyl)-6-[4-(methylsulfonyl)phenyl]spiro[2.4]hepta-4,6-diene (B-122);

4-[6-(4-fluorophenyl)spiro[2.4]hepta-4,6-dien-5-yl]benzenesulfonamide (B-123);

6-(4-fluorophenyl)-2-methoxy-5-[4-(methylsulfonyl)phenyl]-pyridine-3-carbonitrile (B-124);

2-bromo-6-(4-fluorophenyl)-5-[4-(methylsulfonyl)phenyl]-pyridine-3-carbonitrile (B-125);

6-(4-fluorophenyl)-5-[4-(methylsulfonyl)phenyl]-2-phenyl-pyridine-3-carbonitrile (B-126);

4-[2-(4-methylpyridin-2-yl)-4-(trifluoromethyl)-1H-imidazol-1-yl]benzenesulfonamide (B-127);

4-[2-(5-methylpyridin-3-yl)-4-(trifluoromethyl)-1H-imidazol-1-yl]benzenesulfonamide (B-128);

4-[2-(2-methylpyridin-3-yl)-4-(trifluoromethyl)-1H-imidazol-1-yl]benzenesulfonamide (B-129);

3-[1-[4-(methylsulfonyl)phenyl]-4-(trifluoromethyl)-1H-imidazol-2-yl]pyridine (B-130);

2-[1-[4-(methylsulfonyl)phenyl-4-(trifluoromethyl)-1H-imidazol-2-yl]pyridine (B-131);

2-methyl-4-[1-[4-(methylsulfonyl)phenyl-4-(trifluoromethyl)-1H-imidazol-2-yl]pyridine (B-132);

2-methyl-6-[1 -[4-(methylsulfonyl)phenyl-4-(trifluoromethyl)-1H-imidazol-2-yl]pyridine (B-133);

4-[2-(6-methylpyridin-3-yl)-4-(trifluoromethyl)-1H-imidazol-1-yl]benzenesulfonamide (B-134);

2-(3,4-difluorophenyl)-1-[4-(methylsulfonyl)phenyl]-4-(trifluoromethyl)-1H-imidazole (B-135);

4-[2-(4-methylphenyl)-4-(trifluoromethyl)-1H-imidazol-1-yl]benzenesulfonamide (B-136);

2-(4-chlorophenyl)-1-[4-(methylsulfonyl)phenyl]-4-methyl-1H-imidazole (B-137);

2-(4-chlorophenyl)-1-[4-(methylsulfonyl)phenyl]-4-phenyl-1H-imidazole (B-138);

2-(4-chlorophenyl)-4-(4-fluorophenyl)-1-[4-(methylsulfonyl)phenyl]-1H-imidazole (B-139);

2-(3-fluoro-4-methoxyphenyl)-1-[4-(methylsulfonyl)phenyl-4-(trifluoromethyl)-1H-imidazole (B-140);

1-[4-(methylsulfonyl)phenyl]-2-phenyl-4-trifluoromethyl-1H-imidazole (B-141);

2-(4-methylphenyl)-1-[4-(methylsulfonyl)phenyl]-4-trifluoromethyl-1H-imidazole (B-142);

4-[2-(3-chloro-4-methylphenyl)-4-(trifluoromethyl)-1H-imidazol-1-yl]benzenesulfonamide (B-143);

2-(3-fluoro-5-methylphenyl)-1-[4-(methylsulfonyl)phenyl]-4-(trifluoromethyl)-1H-imidazole (B-144);

4-[2-(3-fluoro-5-methylphenyl)-4-(trifluoromethyl)-1H-imidazol-1-yl]benzenesulfonamide (B-145);

2-(3-methylphenyl)-1-[4-(methylsulfonyl)phenyl]-4-trifluoromethyl-1H-imidazole (B-146);

4-[2-(3-methylphenyl)-4-trifluoromethyl-1H-imidazol-1-yl]benzenesulfonamide (B-147);

1-[4-(methylsulfonyl)phenyl]-2-(3-chlorophenyl)-4-trifluoromethyl-1H-imidazole (B-148);

4-[2-(3-chlorophenyl)-4-trifluoromethyl-1H-imidazol-1-yl]benzenesulfonamide (B-149);

4-[2-phenyl-4-trifluoromethyl-1H-imidazol-1-yl]benzenesulfonamide (B-150);

4-[2-(4-methoxy-3-chlorophenyl)-4-trifluoromethyl-1H-imidazol-1-yl]benzenesulfonamide (B-151);

1-allyl-4-(4-fluorophenyl)-3-[4-(methylsulfonyl)phenyl]-5-(trifluoromethyl)-1H-pyrazole (B-152);

4-[1-ethyl-4-(4-fluorophenyl)-5-(trifluoromethyl)-1H-pyrazol-3-yl]benzenesulfonamide (B-153);

N-phenyl-[4-(4-fluorophenyl)-3-[4-(methylsulfonyl)phenyl]-5-(trifluoromethyl)-1H-pyrazol-1-yl]acetamide (B-154);

ethyl [4-(4-fluorophenyl)-3-[4-(methylsulfonyl)phenyl]-5-(trifluoromethyl)-1H-pyrazol-1-yl]acetate (B-155);

4-(4-fluorophenyl)-3-[4-(methylsulfonyl)phenyl]-1-(2-phenylethyl)-1H-pyrazole (B-156);

4-(4-fluorophenyl)-3-[4-(methylsulfonyl)phenyl]-1-(2-phenylethyl)-5-(trifluoromethyl)pyrazole (B-157);

1-ethyl-4-(4-fluorophenyl)-3-[4-(methylsulfonyl)phenyl]-5-(trifluoromethyl)-1H-pyrazole (B-158);

5-(4-fluorophenyl)-4-(4-methylsulfonylphenyl)-2-trifluoromethyl-1H-imidazole (B-159);

4-[4-(methylsulfonyl)phenyl]-5-(2-thiophenyl)-2-(trifluoromethyl)-1H-imidazole (B-160);

5-(4-fluorophenyl)-2-methoxy-4-[4-(methylsulfonyl)phenyl]-6-(trifluoromethyl)pyridine (B-161);

2-ethoxy-5-(4-fluorophenyl)-4-[4-(methylsulfonyl)phenyl]-6-(trifluoromethyl)pyridine (B-162);

5-(4-fluorophenyl)-4-[4-(methylsulfonyl)phenyl]-2-(2-propynyloxy)-6-(trifluoromethyl)pyridine (B-163);

2-bromo-5-(4-fluorophenyl)-4-[4-(methylsulfonyl)phenyl]-6-(trifluoromethyl)pyridine (B-164);

4-[2-(3-chloro-4-methoxyphenyl)-4,5-difluorophenyl]benzenesulfonamide (B-165);

1-(4-fluorophenyl)-2-[4-(methylsulfonyl)phenyl]benzene (B-166);

5-difluoromethyl-4-(4-methylsulfonylphenyl)-3-phenylisoxazole (B-167);

4-[3-ethyl-5-phenylisoxazol-4-yl]benzenesulfonamide (B-168);

4-[5-difluoromethyl-3-phenylisoxazol-4-yl]benzenesulfonamide (B-169);

4-[5-hydroxymethyl-3-phenylisoxazol-4-yl]benzenesulfonamide (B-170);

4-[5-methyl-3-phenyl-isoxazol-4-yl]benzenesulfonamide (B-171);

1-[2-(4-fluorophenyl)cyclopenten-1-yl]-4-(methylsulfonyl)benzene (B-172);

1-[2-(4-fluoro-2-methylphenyl)cyclopenten-1-yl]-4-(methylsulfonyl)benzene (B-173);

1-[2-(4-chlorophenyl)cyclopenten-1-yl]-4-(methylsulfonyl)benzene (B-174);

1-[2-(2,4-dichlorophenyl)cyclopenten-1-yl]-4-(methylsulfonyl)benzene (B-175);

1-[2-(4-trifluoromethylphenyl)cyclopenten-1-yl]-4-(methylsulfonyl)benzene (B-176);

1-[2-(4-methylthiophenyl)cyclopenten-1-yl]-4-(methylsulfonyl)benzene (B-177);

1-[2-(4-fluorophenyl)-4,4-dimethylcyclopenten-1-yl]-4-(methylsulfonyl)benzene (B-178);

4-[2-(4-fluorophenyl)-4,4-dimethylcyclopenten-1-yl]benzenesulfonamide (B-179);

1-[2-(4-chlorophenyl)-4,4-dimethylcyclopenten-1-yl]-4-(methylsulfonyl)benzene (B-180);

4-[2-(4-chlorophenyl)-4,4-dimethylcyclopenten-1-yl]benzenesulfonamide (B-181);

4-[2-(4-fluorophenyl)cyclopenten-1-yl]benzenesulfonamide (B-182);

4-[2-(4-chlorophenyl)cyclopenten-1-yl]benzenesulfonamide (B-183);

1-[2-(4-methoxyphenyl)cyclopenten-1-yl]-4-(methylsulfonyl)benzene (B-184);

1-[2-(2,3-difluorophenyl)cyclopenten-1-yl]-4-(methylsulfonyl)benzene (B-185);

4-[2-(3-fluoro-4-methoxyphenyl)cyclopenten-1-yl]benzenesulfonamide (B-186);

1-[2-(3-chloro-4-methoxyphenyl)cyclopenten-1-yl]-4-(methylsulfonyl)benzene (B-187);

4-[2-(3-chloro-4-fluorophenyl)cyclopenten-1-yl]benzenesulfonamide (B-188);

4-[2-(2-methylpyridin-5-yl)cyclopenten-1-yl]benzenesulfonamide (B-189);

ethyl 2-[4-(4-fluorophenyl)-5-[4-(methylsulfonyl) phenyl]oxazol-2-yl]-2-benzyl-acetate (B-190);

2-[4-(4-fluorophenyl)-5-[4-(methylsulfonyl)phenyl]oxazol-2-yl]acetic acid (B-191);

2-(tert-butyl)-4-(4-fluorophenyl)-5-[4-(methylsulfonyl)phenyl]oxazole (B-192);

4-(4-fluorophenyl)-5-[4-(methylsulfonyl)phenyl]-2-phenyloxazole (B-193);

4-(4-fluorophenyl)-2-methyl-5-[4-(methylsulfonyl)phenyl]oxazole (B-194);

4-[5-(3-fluoro-4-methoxyphenyl)-2-trifluoromethyl-4-oxazolyl]benzenesulfonamide (B-195);

6-chloro-7-(1,1-dimethylethyl)-2-trifluoromethyl-2H-1-benzopyran-3-carboxylic acid (B-196);

6-chloro-8-methyl-2-trifluoromethyl-2H-1-benzopyran-3-carboxylic acid (B-197);

5,5-dimethyl-3-(3-fluorophenyl)-4-methylsulfonyl-2(5H)-furanone (B-198);

6-chloro-2-trifluoromethyl-2H-1-benzothiopyran-3-carboxylic acid (B-199);

4-[5-(4-chlorophenyl)-3-(trifluoromethyl)-1H-pyrazol-1-yl]benzenesulfonamide (B-200);

4-[5-(4-methylphenyl)-3-(trifluoromethyl)-1H-pyrazol-1-yl]benzenesulfonamide (B-201);

4-[5-(3-fluoro-4-methoxyphenyl)-3-(difluoromethyl)-1H-pyrazol-1-yl]benzenesulfonamide (B-202);

3-[1-[4-(methylsulfonyl)phenyl]-4-trifluoromethyl-1H-imidazol-2-yl]pyridine (B-203);

2-methyl-5-[1-[4-(methylsulfonyl)phenyl]-4-trifluoromethyl-1H-imidazol-2-yl]pyridine (B-204);

4-[2-(5-methylpyridin-3-yl)-4-(trifluoromethyl)-1H-imidazol-1-yl]benzenesulfonamide (B-205);

4-[5-methyl-3-phenylisoxazol-4-yl]benzenesulfonamide (B-206);

4-[5-hydroxymethyl-3-phenylisoxazol-4-yl]benzenesulfonamide (B-207);

[2-trifluoromethyl-5-(3,4-difluorophenyl)-4-oxazolyl]benzenesulfonamide (B-208);

4-[2-methyl-4-phenyl-5-oxazolyl]benzenesulfonamide (B-209);

4-[5-(2-fluoro-4-methoxyphenyl)-2-trifluoromethyl-4-oxazolyl]benzenesulfonamide (B-210);

[2-(2-chloro-6-fluoro-phenylamino)-5-methyl-phenyl]-acetic acid or COX 189 (B-211);

N-(4-Nitro-2-phenoxy-phenyl)-methanesulfonamide or nimesulide (B-212);

N-[6-(2,4-difluoro-phenoxy)-1-oxo-indan-5-yl]-methanesulfonamide or flosulide (B-213);

N-[6-(2,4-Difluoro-phenylsulfanyl)-1-oxo-1H-inden-5-yl]-methanesulfonamide, soldium salt or L-745337 (B-214);

N-[5-(4-fluoro-phenylsulfanyl)-thiophen-2-yl]-methanesulfonamide or RWJ-63556 (B-215);

3 -(3,4-Difluoro-phenoxy)-4-(4-methanesulfonyl-phenyl)-5-methyl-5-(2,2,2-trifluoro-ethyl)-5H-furan-2-one or L-784512 or L-784512 (B-216);

(5Z)-2-amino-5-[[3,5-bis(1,1-dimethylethyl)-4-hydroxyphenyl]methylene]-4(5H)-thiazolone or darbufelone (B-217);

CS-502 (B-218);

LAS-34475 (B-219);

LAS-34555 (B-220);

S-33516 (B-221);

SD-8381 (B-222);

L-783003 (B-223);

N-[3-(formylamino)-4-oxo-6-phenoxy-4H-1-benzopyran-7-yl]-methanesulfonamide or T-614 (B-224);

D-1367 (B-225);

L-748731 (B-226);

(6aR,10aR)-3-(1,1-dimethylheptyl)-6a,7,10,10a-tetrahydro-1-hydroxy-6,6-dimethyl-6H-dibenzo[b,d]pyran-9-carboxylic acid or CT3 (B-227);

CGP-28238 (B-228);

4-[[3,5-bis(1,1-dimethylethyl)-4-hydroxyphenyl]methylene]dihydro-2-methyl-2H-1,2-oxazin-3(4H)-one or BF-389 (B-229);

GR-253035 (B-230);

6-dioxo-9H-purin-8-yl-cinnamic acid (B-231); or

S-2474 (B-232);

or an isomer, a pharmaceutically acceptable salt, ester or prodrug thereof, respectively.

In a further preferred embodiment of the invention, the cyclooxygenase inhibitor used in connection with the method(s) of the present invention can be selected from the class of phenylacetic acid derivative cyclooxygenase-2 selective inhibitors represented by the general structure of Formula (III):

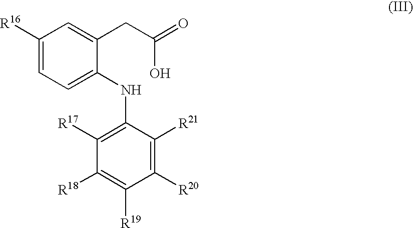

or an isomer, a pharmaceutically acceptable salt, ester, or prodrug thereof; wherein

R¹⁶ is methyl or ethyl;

R¹⁷ is chloro or fluoro;

R¹⁸ is hydrogen or fluoro;

R¹⁹ is hydrogen, fluoro, chloro, methyl, ethyl, methoxy, ethoxy or hydroxy;

R²⁰ is hydrogen or fluoro; and

R²¹ is chloro, fluoro, trifluoromethyl or methyl, provided that R¹⁷, R¹⁸, R¹⁹ and R²⁰ are not all fluoro when R¹⁶ is ethyl and R¹⁹ is H.

A particularly preferred phenylacetic acid derivative cyclooxygenase-2 selective inhibitor used in connection with the method(s) of the present invention is a compound that has the designation of COX 189 (B-211) and that has the structure shown in Formula (III) or an isomer, a pharmaceutically acceptable salt, ester, or prodrug thereof, wherein:

R¹⁶ is ethyl;

R¹⁷ and R¹⁹ are chloro;

R¹⁸ and R²⁰ are hydrogen; and

and R²¹ is methyl.

According to another embodiment, the invention is directed to a method for the treatment of neoplasia disorders comprising administering to a subject in need thereof, a cyclooxygenase-2 (Cox-2) inhibitor in a first amount and thalidomide in a second amount, wherein said first amount together with said second amount is a therapeutically effective amount of said Cox-2 inhibitor and thalidomide, and wherein said Cox-2 inhibitor is represented by Formula (IV):

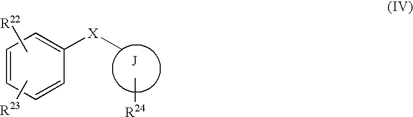

or an isomer, a pharmaceutically acceptable salt, an ester, or a prodrug thereof, wherein:

X is O or S;

J is a carbocycle or a heterocycle;

R²² is NHSO₂CH₃ or F;

R²³ is H, NO₂, or F; and

R²⁴ is H, NHSO₂CH₃, or (SO₂CH₃)C₆H₄.

Further information on the applications of N-(2-cyclohexyloxynitrophenyl)methane sulfonamide (NS-398, CAS RN 123653-11-2), having a structure as shown in formula B-26, have been described by, for example, Yoshimi, N. et al., in Japanese J. Cancer Res., 90(4):406-412 (1999); Falgueyret, J. -P. et al., in Science Spectra, available at: http://www.gbhap.com/Science-_Spectra/20-1-article.htm (Jun. 6, 2001); and Iwata, K. et al., in Jpn. J. Pharmacol., 75(2):191-194 (1997).

An evaluation of the antiinflammatory activity of the cyclooxygenase-2 selective inhibitor, RWJ 63556, in a canine model of inflammation, was described by Kirchner et al., in J Pharmacol Exp Ther 282, 1094-1101 (1997).

According to another embodiment, the Cox-2 inhibitors used in combination with thalidomide have the structural Formula (V):

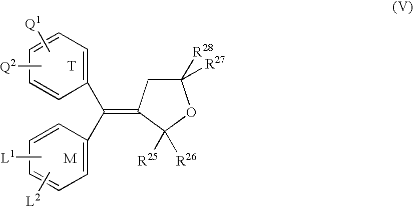

or an isomer, a pharmaceutically acceptable salt, an ester, or a prodrug thereof, wherein:

T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;

Q¹, Q², L¹ or L² are independently hydrogen, halogen, lower alkyl having from 1 to 6 carbon atoms, trifluoromethyl, or lower methoxy having from 1 to 6 carbon atoms; and

at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or,

Q¹ and Q² are methylenedioxy; or

L¹ and L² are methylenedioxy; and

R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,

R²⁵ and R²⁶ are O; or,

R²⁷ and R²⁸ are O; or,

R²⁵, R²⁶, together with the carbon atom to which they are attached, form a saturated hydrocarbon ring having from 3 to 7 carbon atoms; or,

R²⁷, R²⁸, together with the carbon atom to which they are attached, form a saturated hydrocarbon ring having from 3 to 7 carbon atoms.

Particular materials that are included in this family of compounds, and which can serve as the cyclooxygenase-2 selective inhibitor in the present invention, include N-(2-cyclohexyloxynitrophenyl)methane sulfonamide, and (E)-4-[(4-methylphenyl)(tetrahydro-2-oxo-3-furanylidene) methyl] benzenesulfonamide.

The present invention is also directed to a novel method of treating, preventing or inhibiting angiogenesis, wherein said method comprises administering a composition comprising a cyclooxygenase-2 inhibitor or a pharmaceutically acceptable salt, ester or prodrug thereof in a first amount and a thalidomide, thalidomide analog, thalidomide hydrolysis product, thalidomide metabolite or thalidomide precursor in a second amount, wherein said first amount together with said second amount comprises a therapeutically effective amount for the treatment, prevention or inhibition of angiogenesis.

The cyclooxygenase-2 selective inhibitors described previously may be referred to herein collectively as Cox-2 selective inhibitors, or cyclooxygenase-2 selective inhibitors.

Cyclooxygenase-2 selective inhibitors as well as thalidomide that are useful in the present invention can be supplied by any source as long as the combination of drugs is pharmaceutically acceptable. Cyclooxygenase-2-selective inhibitors and thalidomide can be isolated and purified from natural sources or can be synthesized. The combination of the cyclooxygenase-2-selective inhibitor(s) and thalidomide should be of a quality and purity that is conventional in the trade for use in pharmaceutical products.

In the present method, a subject in need of treatment of a neoplasia disorder is treated with an amount of at least one Cox-2 selective inhibitor and an amount of thalidomide, where the amount of the Cox-2 selective inhibitor together with the amount of thalidomide is sufficient to constitute a therapeutically effective amount for treating said neoplasia disorder.

As used herein, an “effective amount” or “therapeutically effective amount” means the dose or effective amount to be administered to a patient and the frequency of administration to the subject which is sufficient to obtain a therapeutic effect as readily determined by one of ordinary skill in the art, by the use of known techniques and by observing results obtained under analogous circumstances. The dose or effective amount to be administered to a patient and the frequency of administration to the subject can be readily determined by one of ordinary skill in the art by the use of known techniques and by observing results obtained under analogous circumstances. In determining the effective amount or dose, a number of factors are considered by the attending diagnostician, including but not limited to, the potency and duration of action of the compounds used; the nature and severity of the illness to be treated as well as on the sex, age, weight, general health and individual responsiveness of the patient to be treated, and other relevant circumstances.

The phrase “therapeutically effective” indicates the capability of a combination of agents to prevent, or reduce the severity of, the disorder or its undesirable symptoms, while avoiding adverse side effects typically associated with alternative therapies.

Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

The amounts of the Cox-2 selective inhibitor and thalidomide that are used in the subject method may be amounts that, together, are sufficient to constitute an effective amount for neoplasia treatment, prevention or inhibition. In the present method, the amount of Cox-2 selective inhibitor that is used in the novel method of treatment preferably ranges from about 0.001 to about 100 milligrams per day per kilogram of body weight of the subject (mg/day.kg), more preferably from about 0.05 to about 50 mg/day.kg, even more preferably from about 1 to about 20 mg/day.kg.

When the Cox-2 selective inhibitor comprises rofecoxib, it is preferred that the amount used is within a range of from about 0.15 to about 1.0 mg/day.kg, and even more preferably from about 0.18 to about 0.4 mg/day.kg.

When the Cox-2 selective inhibitor comprises etoricoxib, it is preferred that the amount used is within a range of from about 0.5 to about 5 mg/day.kg, and even more preferably from about 0.8 to about 4 mg/day.kg.

When the Cox-2 selective inhibitor comprises celecoxib, it is preferred that the amount used is within a range of from about 1 to about 20 mg/day.kg, even more preferably from about 1.4 to about 8.6 mg/day-kg, and yet more preferably from about 2 to about 3 mg/day-kg.

When the Cox-2 selective inhibitor comprises valdecoxib, it is preferred that the amount used is within a range of from about 0.1 to about 5 mg/day-kg, and even more preferably from about 0.8 to about 4 mg/day-kg.

When the Cox-2 selective inhibitor comprises parecoxib, it is preferred that the amount used is within a range of from about 0.1 to about 5 mg/day.kg, and even more preferably from about 1 to about 3 mg/day.kg.

In terms of absolute daily dosages, when the Cox-2 selective inhibitor comprises rofecoxib, it is preferred that the amount used is from about 10 to about 75 mg/day, more preferably from about 12.5 to about 50 mg/day.

When the Cox-2 selective inhibitor comprises etoricoxib, it is preferred that the amount used is from about 50 to about 100 mg/day, more preferably from about 60 to about 90 mg/day. When the Cox-2 selective inhibitor comprises celecoxib, it is preferred that the amount used is from about 100 to about 1000 mg/day, more preferably from about 200 to about 800 mg/day. When the Cox-2 selective inhibitor comprises valdecoxib, it is preferred that the amount used is from about 5 to about 100 mg/day, more preferably from about 10 to about 60 mg/day. When the Cox-2 selective inhibitor comprises parecoxib, it is preferred that the amount used is within a range of from about 10 to about 100 mg/day, more preferably from about 20 to about 80 mg/day.

It is preferred that the amount of thalidomide that is used in combination with a COX-2 selective inhibitor for a single dosage of treatment is within range of from about 1 to about 600 milligrams per day (mg/day), preferably of from about 100 to about 500 mg/day, and more preferably from about 200 to about 400 mg/day. The frequency of dose will depend upon the half-life of thalidomide or an analog, hydrolysis product, metabolite, or precursor thereof. If the thalidomide or analog, hydrolysis product, metabolite, or precursor thereof has a short half-life (e.g. from about 2 to 10 hours) it may be necessary to give one or more doses per day. Alternatively, it the thalidomide or analog, hydrolysis product, metabolite, or precursor thereof has a long half-life (e.g. from about 2 to about 15 days) it may only be necessary to give a dosage once per day, per week, or even once every 1 or 2 months. A preferred dosage rate is to administer the dosage amounts described above to a subject once per day. It will be apparent to those skilled in the art that it is possible, and perhaps desirable, to combine various times and methods of administration in the practice of the present methods.

In another embodiment, the combination therapy of thalidomide and a COX-2 selective inhibitor may be administered alone or in conjunction with a standard tumor therapy, such as chemotherapy or radiation therapy. It is preferred that the combination therapy of thalidomide and a COX-2 selective inhibitor be administered in combination with a standard cancer therapy, preferably, chemotherapy or radiation therapy. While not wishing to be bound by any theory, the effect of the administration of a pharmaceutical compound comprising thalidomide and a COX-2 selective inhibitor to inhibit neoplasia by preferably inhibiting or preventing tumor growth, is related to the ability of the pharmaceutical compound to inhibit endothelial cell proliferation and hence new blood vessel formation. Such reduction of vascular supply works best when a tumor is significantly reduced in mass after standard chemotherapy or radiation therapy. As such, in a preferred embodiment, the pharmaceutical compounds of the present invention are administered in conjunction with the standard antitumor therapy and, in addition, can be administered on a continuing basis after the standard antitumor therapy. In this way the tumor will grow back slower while the patient is recovering from the side effects of the standard therapy. Chemotherapy or radiation therapy can then be repeated along with the continuation of the administration of the compound comprising thalidomide and a COX-2 selective inhibitor. Preferably, the effect of this continuation of combination therapy is that the pharmaceutical compound will be effective in slowing down vascular supply to an already weakened tumor until it is essentially eradicated.

The Cox-2 selective inhibitor(s) and thalidomide that are described above can be provided in a therapeutic composition so that the preferred amounts thereof is/are supplied by a single dosage, a single capsule for example, or, by up to four, or more, single dosage forms.

In one embodiment of the invention, the Cox-2 inhibitor(s) and thalidomide, thalidomide analog, thalidomide hydrolysis product, thalidomide metabolite or thalidomide precursor may be administered substantially simultaneously, meaning that both agents may be provided in a single dosage, for example by mixing the agents and incorporating the mixture into a single capsule. Otherwise, the Cox-2 inhibitor(s) and thalidomide may be administered substantially simultaneously by administration in separate dosages within a short time period, for example within 5 minutes or less. Alternatively, the Cox-2 inhibitor(s) and thalidomide may be administered sequentially, meaning that separate dosages, and possibly even separate dosage forms of the Cox-2 inhibitor(s) and thalidomide may be administered at separate times, for example on a staggered schedule but with equal frequency of administration of the Cox-2 inhibitor(s) and thalidomide. Of course, it is also possible that the Cox-2 inhibitor(s) may be administered either more or less frequently than thalidomide. In any case, it is preferable that, among successive time periods of a sufficient length, for example one day, the weight ratio of the Cox-2 inhibitor(s) administered to the weight ratio of thalidomide administered remains constant.

The term “pharmacologically effective amount” shall mean that amount of a drug or pharmaceutical agent that will elicit the biological or medical response of a tissue, system, animal or human that is being sought by a researcher or clinician. This amount can be a therapeutically effective amount.

The term “pharmaceutically acceptable” is used herein to mean that the modified noun is appropriate for use in a pharmaceutical product. Pharmaceutically acceptable cations include metallic ions and organic ions. More preferred metallic ions include, but are not limited to, appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include, without limitation, hydrochloric acid, hydroiodic acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

Also included in connection with use of the method(s) of the present invention are the isomeric forms and tautomers and the pharmaceutically-acceptable salts of the cyclooxygenase-2 selective inhibitors. Isomers of Cox-2 inhibitors include their diastereomers, enantiomers, and racemates as well as their structural to isomers. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaaminosulfonic, algenic, β-hydroxybutyric, galactaric, and galacturonic acids.

Suitable pharmaceutically-acceptable base addition salts of compounds used in connection with the method(s) of the present invention include metallic ion salts and organic ion salts. More preferred metallic ion salts include, but are not limited to, appropriate alkali metal (group Ia) salts, alkaline earth metal (group Ia) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention. Pharmaceutically acceptable esters include, but are not limited to, the alkyl esters of the Cox-2 inhibitors.

The terms “treating” or “to treat” means to alleviate symptoms, eliminate the causation either on a temporary or permanent basis, or to prevent or slow the appearance of symptoms. The term “treatment” includes alleviation, elimination of causation of or prevention of undesirable symptoms associated with a neoplasia disorder. Besides being useful for human treatment, these combinations are also useful for treatment of mammals, including horses, dogs, cats, rats, mice, sheep, pigs, etc.

The term “subject” for purposes of treatment includes any human or animal subject who is in need of the treatment, prevention or inhibition of a neoplasia disorder. The subject is typically a human subject.

For methods of prevention, the subject is any human or animal subject, and preferably is a subject that is in need of prevention and/or treatment of a neoplasia disorder. The subject may be a human subject who is at risk for neoplasia. The subject may be at risk for neoplasia due to genetic predisposition, lifestyle, diet, exposure to disorder-causing agents, exposure to pathogenic agents and the like.

In connection with the inventive method, the Cox-2 pharmaceutical composition(s) and thalidomide, thalidomide analog, thalidomide hydrolysis product, thalidomide metabolite or thalidomide precursor may be administered enterally and parenterally. Parenteral administration includes subcutaneous, intramuscular, intradermal, intramammary, intravenous, and other administrative methods known in the art. Enteral administration includes solution, tablets, sustained release capsules, enteric coated capsules, and syrups. When administered, the pharmaceutical composition may be at or near body temperature.

The phrase “administration” in defining the use of both a cyclooxygenase-2 inhibitor agent and thalidomide is intended to embrace administration of each agent in a manner and in a regimen that will provide beneficial effects of the drug combination therapy, and is intended as well to embrace co-administration of 2 or more of the Cox-2 agents in a substantially simultaneous manner and/or 2 or more of the thalidomide, thalidomide analog, thalidomide hydrolysis product, thalidomide metabolite or thalidomide precursor in a substantially simultaneous manner, such as in a single capsule or dosage device having a fixed ratio of these active agents or in multiple, separate capsules or dosage devices for each agent, where the separate capsules or dosage devices can be taken together contemporaneously, or taken within a period of time sufficient to receive a beneficial effect from the constituent Cox-2 agent and thalidomide used in combination.

The phrases “therapeutically-effective” and “effective for the treatment, prevention, or inhibition”, are intended to qualify the amount of each Cox-2 agent and thalidomide for use in the Cox-2 therapy which will achieve the goal of reduction of the severity and/or frequency of incidence of neoplasia associated symptoms, while avoiding adverse side effects typically associated with alternative therapies.

In particular, the pharmaceutical composition of one or more Cox-2 inhibitors and thalidomide in connection with the method(s) of the present invention can be administered orally, for example, as tablets, coated tablets, dragees, troches, lozenges, gums, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, maize starch, or alginic acid; binding agents, for example starch, gelatin or acacia, and lubricating agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Aqueous suspensions can be produced that contain the active materials in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing the novel combination may be formulated with sweetening agents, for example glycerol, sorbitol or sucrose. Such formulations may also contain a demulcent, a preservative and flavoring and coloring agents.

The subject pharmaceutical composition of Cox-2 inhibitor(s) and thalidomide in connection with the present inventive method can also be administered parenterally, either subcutaneously, or intravenously, or intramuscularly, or intrastemally, or by infusion techniques, in the form of sterile injectable aqueous or olagenous suspensions. Such suspensions may be formulated according to the known art using those suitable dispersing of wetting agents and suspending agents which have been mentioned above, or other acceptable agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, n-3 polyunsaturated fatty acids may find use in the preparation of injectables.

The subject pharmaceutical composition of Cox-2 inhibitor(s) and thalidomide in connection with the present inventive method can also be administered by inhalation, in the form of aerosols or solutions for nebulizers, or rectally, in the form of suppositories prepared by mixing the drug with a suitable non-irritating excipient which is solid at ordinary temperature but liquid at the rectal temperature and will therefore melt in the rectum to release the drug. Such materials are cocoa butter and poly-ethylene glycols.

The pharmaceutical compositions of Cox-2 inhibitor(s) and thalidomide in connection with the present inventive method can also be administered topically, in the form of patches, creams, ointments, jellies, collyriums, solutions or suspensions. Of course, the compositions of the present invention can be administered by routes of administration other than topical administration. Also, as mentioned above, the Cox-2 inhibitor(s) and thalidomide may be administered separately, with each agent administered by any of the above mentioned administration routes. For example, the Cox-2 inhibitor(s) may be administered orally in any or the above mentioned forms (e.g. in capsule form) while the thalidomide is administered topically (e.g. as a cream).

Daily dosages can vary within wide limits and will be adjusted to the individual requirements in each particular case. In general, for administration to adults, an appropriate daily dosage has been described above, although the limits that were identified as being preferred may be exceeded if expedient. The daily dosage can be administered as a single dosage or in divided dosages.

Various delivery systems include capsules, tablets, and gelatin capsules, for example.

Other embodiments within the scope of the embodiments herein will be apparent to one skilled in the art from consideration of the specification or practice of the invention as disclosed herein. It is intended that the specification be considered to be exemplary only, with the scope and spirit of the invention being indicated by the embodiments.

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained.

As various changes could be made in the above methods and compositions without departing from the scope of the invention, it is intended that all matter contained in this application shall be interpreted as illustrative and not in a limiting sense.

1. A composition for the treatment, prevention or inhibition of neoplasia disorder in a subject in need of such treatment comprising a cyclooxygenase-2 inhibitor or a pharmaceutically acceptable salt, ester or prodrug thereof in a first amount and a thalidomide, thalidomide analog, thalidomide hydrolysis product, thalidomide metabolite or thalidomide precursor in a second amount, wherein said first amount together with said second amount comprises a therapeutically effective amount for the treatment, prevention or inhibition of neoplasia disorder in said subject.

2. The composition of claim 1, wherein said Cox-2 inhibitor or isomer, pharmaceutically acceptable salt, ester, or prodrug thereof has a Cox-2 IC₅₀ of less than about 5 μmol/L.

3. The composition of claim 2, wherein said Cox-2 inhibitor or isomer, pharmaceutically acceptable salt, ester, or prodrug thereof has a selectivity ratio of Cox-2 inhibition to Cox-1 inhibition of at least about 1.5.

4. The composition of claim 3, wherein said Cox-2 inhibitor or isomer, pharmceutically acceptable salt, ester, or prodrug thereof has a Cox-2 IC₅₀ of less than about 1 μmol/L and a selectivity ratio of Cox-2 inhibition to Cox-1 inhibition of at least about 100.

5. The composition of claim 1, wherein said Cox-2 inhibitor or isomer, pharmaceutically acceptable salt, ester, or prodrug thereof has a Cox-1 IC₅₀ of at least about 1 μmol/L.

6. The composition of claim 5, wherein said Cox-2 inhibitor or isomer, pharmaceutically acceptable salt, ester, or prodrug thereof has a Cox-1 IC₅₀ of at least about 20 μmol/L.

7. The composition of claim 1, wherein said subject is an animal.

8. The composition of claim 7, wherein said subject is a human.

9. The composition of claim 1, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered enterally or parenterally in one or more doses per day.

10. The composition of claim 1, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered substantially simultaneously.

11. The composition of claim 1, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered sequentially.

12. The composition of claim 1, wherein the neoplasia disorder is a tumor growth.

13. The composition of claim 12, wherein the tumor growth is a malignant tumor growth or a benign tumor growth.

14. The composition of claim 13, wherein the malignant tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

15. The composition of claim 13, wherein the malignant growth is a viral-related cancer.

16. The composition of claim 15, wherein the viral-related cancer includes cervical cancer, T-cell leukemia, lymphoma, and Kaposi's sarcoma.

17. The composition of claim 13, wherein the benign tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

18. The composition of claim 17, wherein the benign tumor growth is a fibroid tumor, an endometriosis, or a cyst.

19. A composition for the treatment, prevention or inhibition of neoplasia disorder in a subject in need of such treatment comprising a cyclooxygenase-2 inhibitor or a pharmaceutically acceptable salt, ester or prodrug thereof selected from the group consisting of substituted benzothiopyrans, dihydroquinolines, and dihydronaphtalenes in a first amount and a thalidomide, thalidomide analog, thalidomide hydrolysis product, thalidomide metabolite or thalidomide precursor in a second amount, wherein said first amount together with said second amount comprises a therapeutically effective amount for the treatment, prevention or inhibition of neoplasia disorder in said subject.

20. A composition for treating neoplasia disorder comprising administering to a subject in need thereof, a cyclooxygenase-2 (Cox-2) inhibitor in a first amount and a thalidomide, thalidomide analog, thalidomide hydrolysis product, thalidomide metabolite or thalidomide precursor in a second amount, wherein said first amount together with said second amount is a therapeutically effective amount of said Cox-2 inhibitor and said thalidomide, thalidomide analog, thalidomide hydrolysis product, thalidomide metabolite or thalidomide precursor, and wherein said Cox-2 inhibitor is represented by Formula (I): 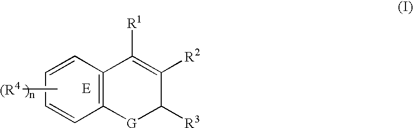
or an isomer, a pharmaceutically acceptable salt, ester, or prodrug thereof; wherein:
G is O, S or NRa;
R^{a} is alkyl;
R¹ is H or aryl;
R² is carboxyl, aminocarbonyl, alkylsulfonylaminocarbonyl or alkoxycarbonyl;
R³ is haloalkyl, alkyl, aralkyl, cycloalkyl or aryl optionally and independently substituted with one or more radicals selected from alkylthio, nitro and alkylsulfonyl; n is an integer which is 1, 2, 3, or 4; and
each R⁴ is independently H, halo, alkyl, aryl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, mono- or dialkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, aralkylcarbonyl, heteroarylcarbonyl, arylcarbonyl, aminocarbonyl, alkylcarbonyl, aryl, or heteroaryl; wherein said aryl and heteroaryl radicals are optionally and independently substituted with one or more radicals which are alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy or alkylthio; or wherein R⁴ together with the atoms to which R⁴ is attached and the remainder of ring E forms a naphthyl radical.

21. The composition of claim 20, wherein:
G is O or S;
R² is carboxyl, lower alkyl, lower aralkyl and lower alkoxycarbonyl;
R³ is lower haloalkyl, lower cycloalkyl and phenyl; and each of one or more R⁴ is independently H, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, lower aralkylcarbonyl, lower alkylcarbonyl, and phenyl optionally and independently substituted with one or more radicals selected from the group consisting of alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy or alkylthio; or wherein R⁴ together with the atoms to which R⁴ is attached and the remainder of ring E forms a naphthyl radical.

22. The composition of claim 21, wherein:
R² is carboxyl;
R³ is lower haloalkyl; and
each of one or more R⁴ is independently H, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, or lower alkylcarbonyl;
or wherein R⁴ together with the atoms to which R⁴ is attached and the remainder of ring E forms a naphthyl radical.

23. The composition of claim 22, wherein:
said lower haloalkyl R³ is fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, or trifluoromethyl; and each or one or more R⁴ is independently H, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, benzylaminosulfonyl, N-ethylsulfonyl, 2,2-dimethylethylaminosulfonyl, N,N-dimethylaminosulfonyl, isopropylaminosulfonyl, N-(2-methylpropyl)aminosulfonyl, N-morpholinosulfonyl, methylsulfonyl, benzylcarbonyl, 2,2-dimethylpropylcarbonyl, phenylacetyl, or phenyl;
or wherein R⁴ together with the atoms to which R⁴ is attached and the remainder of the ring E forms a naphthyl radical.

24. The composition of claim 23, wherein:
R³ is trifluoromethyl or pentafluoroethyl; and
each of one or more R⁴ is independently H, chloro, fluoro, bromo, iodo, methyl, ethyl, isopropyl, tert-butyl, methoxy, trifluoromethyl, trifluoromethoxy, N,N-diethylamino, N-phenylmethylaminosulfonyl, N-phenylethylaminosulfonyl, N-(2-furylmethyl)aminosulfonyl, N,N-dimethylaminosulfonyl, N-methylaminosulfonyl, benzylaminosulfonyl, N-(2,2-dimethylethyl)aminosulfonyl, isopropylaminosulfonyl, dimethylaminosulfonyl, 2-methylpropylaminosulfonyl, N-morpholinosulfonyl, methylsulfonyl, benzylcarbonyl, or phenyl;
or wherein R⁴ together with the atoms to which R⁴ is attached and the remainder of ring E forms a naphthyl radical.

25. The composition of claim 24, wherein:
R³ is trifluoromethyl or pentafluoroethyl;
each of one or more R⁴ is independently H, methyl, ethyl, isopropyl, tert-butyl, chloro, bromo, fluoro, iodo, methyl, tert-butyl, trifluoromethoxy, methoxy, benzylcarbonyl, dimethylaminosulfonyl, isopropylaminosulfonyl, N-methylaminosulfonyl, benzylaminosulfonyl, phenylethylaminosulfonyl, methylpropylaminosulfonyl, methylsulfonyl, morpholinosulfonyl, N,N-diethylamino, or phenyl.

26. The composition of claim 20, wherein said first amount is from about 0.001 to about 100 mg/day per kg of body weight of said subject and said second amount is from about 1 to about 600 mg/day per kg of body weight of said subject.

27. The composition of claim 26, wherein said first amount is from about 0.5 to about 50 mg/day per kg of body weight of said subject and said second amount is from about 100 to about 500 mg/day per kg of body weight of said subject.

28. The composition of claim 27, wherein said first amount is from about 1 to about 20 mg/day per kg of body weight of said subject and said second amount is from about 200 to about 400 mg/day per kg of body weight of said subject.

29. The composition of claim 20, wherein said subject is an animal.

30. The composition of claim 29, wherein said subject is a human.

31. The composition of claim 20, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered enterally or parenterally in one or more doses per day.

32. The composition of claim 20, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered substantially simultaneously.

33. The composition of claim 20, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered sequentially.

34. The composition of claim 20, wherein the neoplasia disorder is a tumor growth.

35. The composition of claim 34, wherein the tumor growth is a malignant tumor growth or a benign tumor growth.

36. The composition of claim 35, wherein the malignant tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

37. The composition of claim 35, wherein the malignant growth is a viral-related cancer.

38. The composition of claim 37, wherein the viral-related cancer includes cervical cancer, T-cell leukemia, lymphoma, and Kaposi's sarcoma.

39. The composition of claim 35, wherein the benign tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

40. The composition of claim 39, wherein the benign tumor growth is a fibroid tumor, an endometriosis, or a cyst.

41. A composition for the treatment, prevention or inhibition of neoplasia disorder in a subject in need of such treatment comprising a cyclooxygenase-2 inhibitor or a pharmaceutically acceptable salt, ester or prodrug thereof selected from the group consisting of tricylic Cox-2 inhibitors in a first amount and a thalidomide, thalidomide analog, thalidomide hydrolysis product, thalidomide metabolite or thalidomide precursor in a second amount, wherein said first amount together with said second amount comprises a therapeutically effective amount for the treatment, prevention or inhibition of neoplasia disorder in said subject.

42. A composition for treating neoplasia disorder comprising administering, to a subject in need thereof, a cyclooxygenase-2 (Cox-2) inhibitor in a first amount and a thalidomide, thalidomide analog, thalidomide hydrolysis product, thalidomide metabolite or thalidomide precursor in a second amount, wherein said first amount together with said second amount is a therapeutically effective amount of said Cox-2 inhibitor and said thalidomide, thalidomide analog, thalidomide hydrolysis product, thalidomide metabolite or thalidomide precursor, and wherein said Cox-2 inhibitor is represented by Formula (II): 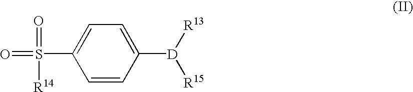
or an isomer, a pharmaceutically acceptable salt, ester, or prodrug thereof, wherein:
D is a partially unsaturated or saturated heterocyclyl ring or a partially unsaturated or saturated carbocyclic ring;
R¹³ is heterocyclyl, cycloalkyl, cycloalkenyl and aryl, wherein R¹³ is optionally substituted at a substitutable position with one or more radicals which are alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy or alkylthio;
R¹⁴ is methyl or amino; and
R¹⁵ is H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, or N-alkyl-N-arylaminosulfonyl.

43. The composition of claim 42, wherein said Cox-2 inhibitor or isomer, pharmaceutically acceptable salt, ester, or prodrug thereof has a Cox-2 IC₅₀ of less than about 5 μmol/L.

44. The composition of claim 43, wherein said Cox-2 inhibitor or isomer, pharmaceutically acceptable salt, ester, or prodrug thereof has a selectivity ratio of Cox-2 inhibition to Cox-1 inhibition of at least about 1.5.

45. The composition of claim 44, wherein said Cox-2 inhibitor or isomer, pharmceutically acceptable salt, ester, or prodrug thereof has a Cox-2 IC₅₀ of less than about 1 μmol/L and a selectivity ratio of Cox-2 inhibition to Cox-1 inhibition of at least about 100.

46. The composition of claim 42, wherein said Cox-2 inhibitor or isomer, pharmaceutically acceptable salt, ester, or prodrug thereof has a Cox-1 IC₅₀ of at least about 1 μmol/L.

47. The composition of claim 46, wherein said Cox-2 inhibitor or isomer, pharmaceutically acceptable salt, ester, or prodrug thereof has a Cox-1 IC₅₀ of at least about 20 μmol/L.

48. The composition of claim 42, wherein said first amount is from about 0.001 to about 100 mg/day per kg of body weight of said subject and said second amount is from about 1 to about 600 mg/day per kg of body weight of said subject.

49. The composition of claim 48, wherein said first amount is from about 0.5 to about 50 mg/day per kg of body weight of said subject and said second amount is from about 100 to about 500 mg/day per kg of body weight of said subject.

50. The composition of claim 49, wherein said first amount is from about 1 to about 20 mg/day per kg of body weight of said subject and said second amount is from about 200 to about 400 mg/day per kg of body weight of said subject.

51. The composition of claim 42, wherein said subject is an animal.

52. The composition of claim 51, wherein said subject is a human.

53. The composition of claim 42, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered enterally or parenterally in one or more doses per day.

54. The composition of claim 42, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered substantially simultaneously.

55. The composition of claim 42, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered sequentially.

56. The composition of claim 42, wherein the neoplasia disorder is a tumor growth.

57. The composition of claim 56, wherein the tumor growth is a malignant tumor growth or a benign tumor growth.

58. The composition of claim 57, wherein the malignant tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

59. The composition of claim 57, wherein the malignant growth is a viral-related cancer.

60. The composition of claim 59, wherein the viral-related cancer includes cervical cancer, T-cell leukemia, lymphoma, and Kaposi's sarcoma.

61. The composition of claim 57, wherein the benign tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

62. The composition of claim 61, wherein the benign tumor growth is a fibroid tumor, an endometriosis, or a cyst.

63. A composition for the treatment, prevention or inhibition of neoplasia disorder in a subject in need of such treatment comprising a cyclooxygenase-2 inhibitor or a pharmaceutically acceptable salt, ester or prodrug thereof selected from the group consisting of phenylacetic acid derivatives in a first amount and a thalidomide, thalidomide analog, thalidomide hydrolysis product, thalidomide metabolite or thalidomide precursor in a second amount, wherein said first amount together with said second amount comprises a therapeutically effective amount for the treatment, prevention or inhibition of neoplasia disorder in said subject.

64. A composition for treating neoplasia disorder comprising administering, to a subject in need thereof, a cyclooxygenase-2 (Cox-2) inhibitor in a first amount and a thalidomide, thalidomide analog, thalidomide hydrolysis product, thalidomide metabolite or thalidomide precursor in a second amount, wherein said first amount together with said second amount is a therapeutically effective amount of said Cox-2 inhibitor and said thalidomide, thalidomide analog, thalidomide hydrolysis product, thalidomide metabolite or thalidomide precursor, and wherein said Cox-2 inhibitor is represented by Formula (III): 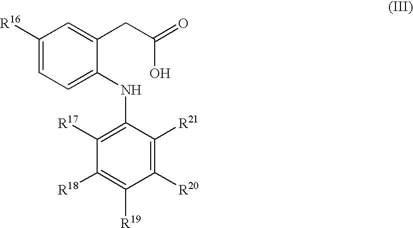
or an isomer, a pharmaceutically acceptable salt, ester, or prodrug thereof, wherein:
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl,
provided that R¹⁷, R¹⁸, R¹⁹ and R²⁰ are not all fluoro when R¹⁶ is ethyl and R¹⁹ is H.

65. The composition of claim 64, wherein:
R¹⁶ is ethyl; R¹⁷ and R¹⁹ are chloro; R¹⁸ and R²⁰ are hydrogen; and R²¹ is methyl.

66. The composition of claim 64, wherein said Cox-2 inhibitor or isomer, pharmaceutically acceptable salt, ester, or prodrug thereof has a Cox-2 IC₅₀ of less than about 5 μmol/L.

67. The composition of claim 66, wherein said Cox-2 inhibitor or isomer, pharmaceutically acceptable salt, ester, or prodrug thereof has a selectivity ratio of Cox-2 inhibition to Cox-1 inhibition of at least about 1.5.

68. The composition of claim 67, wherein said Cox-2 inhibitor or isomer, pharmceutically acceptable salt, ester, or prodrug thereof has a Cox-2 IC₅₀ of less than about 1 μmol/L and a selectivity ratio of Cox-2 inhibition to Cox-1 inhibition of at least about 100.

69. The composition of claim 64, wherein said Cox-2 inhibitor or isomer, pharmaceutically acceptable salt, ester, or prodrug thereof has a Cox-1 IC₅₀ of at least about 1 μmol/L.

70. The composition of claim 69, wherein said Cox-2 inhibitor or isomer, pharmaceutically acceptable salt, ester, or prodrug thereof has a Cox-1 IC₅₀ of at least about 20 μmol/L.

71. The composition of claim 64, wherein said first amount is from about 0.001 to about 100 mg/day per kg of body weight of said subject and said second amount is from about 1 to about 600 mg/day per kg of body weight of said subject.

72. The composition of claim 71, wherein said first amount is from about 0.5 to about 50 mg/day per kg of body weight of said subject and said second amount is from about 100 to about 500 mg/day per kg of body weight of said subject.

73. The composition of claim 72, wherein said first amount is from about 1 to about 20 mg/day per kg of body weight of said subject and said second amount is from about 200 to about 400 mg/day per kg of body weight of said subject.

74. The composition of claim 64, wherein said subject is an animal.

75. The composition of claim 74, wherein said subject is a human.

76. The composition of claim 64, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered enterally or parenterally in one or more doses per day.

77. The composition of claim 64, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered substantially simultaneously.

78. The composition of claim 64, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered sequentially.

79. The composition of claim 64, wherein the neoplasia disorder is a tumor growth.

80. The composition of claim 79, wherein the tumor growth is a malignant tumor growth or a benign tumor growth.

81. The composition of claim 80, wherein the malignant tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

82. The composition of claim 80, wherein the malignant growth is a viral-related cancer.

83. The composition of claim 82, wherein the viral-related cancer includes cervical cancer, T-cell leukemia, lymphoma, and Kaposi's sarcoma.

84. The composition of claim 80, wherein the benign tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

85. The composition of claim 84, wherein the benign tumor growth is a fibroid tumor, an endometriosis, or a cyst.

86. A composition for treating neoplasia disorder comprising administering, to a subject in need thereof, a cyclooxygenase-2 (Cox-2) inhibitor in a first amount and a thalidomide, thalidomide analog, thalidomide hydrolysis product, thalidomide metabolite or thalidomide precursor in a second amount, wherein said first amount together with said second amount is a therapeutically effective amount of said Cox-2 inhibitor and said thalidomide, thalidomide analog, thalidomide hydrolysis product, thalidomide metabolite or thalidomide precursor, and wherein said Cox-2 inhibitor is represented by Formula (IV): 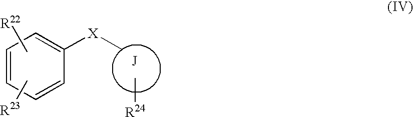
or an isomer, a pharmaceutically acceptable salt, ester, or prodrug thereof, wherein:
X is O or S;
J is a carbocycle or a heterocycle;
R²² is NHSO₂CH₃ or F;
R²³ is H, NO₂, or F; and
R²⁴ is H, NHSO₂CH₃, or (SO₂CH₃)C₆H₄.

87. The composition of claim 86 wherein said Cox-2 inhibitor is nimesulide (B-212), flosulide (B-213), NS-398 (B-26), L-745337 (B-214), RWJ-63556 (B-215), or L-784512 (B-216).

88. The composition of claim 86, wherein said Cox-2 inhibitor or isomer, pharmaceutically acceptable salt, ester, or prodrug thereof has a Cox-2 IC₅₀ of less than about 5 μmol/L.

89. The composition of claim 88, wherein said Cox-2 inhibitor or isomer, pharmaceutically acceptable salt, ester, or prodrug thereof has a selectivity ratio of Cox-2 inhibition to Cox-1 inhibition of at least about 1.5.

90. The composition of claim 89, wherein said Cox-2 inhibitor or isomer, pharmceutically acceptable salt, ester, or prodrug thereof has a Cox-2 IC₅₀ of less than about 1 μmol/L and a selectivity ratio of Cox-2 inhibition to Cox-1 inhibition of at least about 100.

91. The composition of claim 86, wherein said Cox-2 inhibitor or isomer, pharmaceutically acceptable salt, ester, or prodrug thereof has a Cox-1 IC₅₀ of at least about 1 μmol/L.

92. The composition of claim 91, wherein said Cox-2 inhibitor or isomer, pharmaceutically acceptable salt, ester, or prodrug thereof has a Cox-1 IC₅₀ of at least about 20 μmol/L.

93. The composition of claim 86, wherein said first amount is from about 0.001 to about 100 mg/day per kg of body weight of said subject and said second amount is from about 1 to about 600 mg/day per kg of body weight of said subject.

94. The composition of claim 93, wherein said first amount is from about 0.5 to about 50 mg/day per kg of body weight of said subject and said second amount is from about 100 to about 500 mg/day per kg of body weight of said subject.

95. The composition of claim 94, wherein said first amount is from about 1 to about 20 mg/day per kg of body weight of said subject and said second amount is from about 200 to about 400 mg/day per kg of body weight of said subject.

96. The composition of claim 86, wherein said subject is an animal.

97. The composition of claim 96, wherein said subject is a human.

98. The composition of claim 86, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered enterally or parenterally in one or more doses per day.

99. The composition of claim 86, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered substantially simultaneously.

100. The composition of claim 86, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered sequentially.

101. The composition of claim 86, wherein the neoplasia disorder is a tumor growth.

102. The composition of claim 101, wherein the tumor growth is a malignant tumor growth or a benign tumor growth.

103. The composition of claim 102, wherein the malignant tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

104. The composition of claim 102, wherein the malignant growth is a viral-related cancer.

105. The composition of claim 104, wherein the viral-related cancer includes cervical cancer, T-cell leukemia, lymphoma, and Kaposi's sarcoma.

106. The composition of claim 102, wherein the benign tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

107. The composition of claim 106, wherein the benign tumor growth is a fibroid tumor, an endometriosis, or a cyst.

108. A composition for the treatment, prevention or inhibition of neoplasia disorder in a subject in need of such treatment comprising a cyclooxygenase-2 inhibitor or a pharmaceutically acceptable salt, ester or prodrug thereof selected from the group consisting of diarylmethyldenefuran and diarylmethyldenefuran derivatives in a first amount and a thalidomide, thalidomide analog, thalidomide hydrolysis product, thalidomide metabolite or thalidomide precursor in a second amount, wherein said first amount together with said second amount comprises a therapeutically effective amount for the treatment, prevention or inhibition of neoplasia disorder in said subject.

109. A composition for treating neoplasia disorder comprising administering, to a subject in need thereof, a cyclooxygenase-2 (Cox-2) inhibitor in a first amount and a thalidomide, thalidomide analog, thalidomide hydrolysis product, thalidomide metabolite or thalidomide precursor in a second amount, wherein said first amount together with said second amount is a therapeutically effective amount of said Cox-2 inhibitor and said thalidomide, thalidomide analog, thalidomide hydrolysis product, thalidomide metabolite or thalidomide precursor, and wherein said Cox-2 inhibitor is represented by Formula (V): 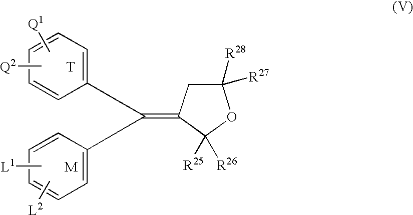
or an isomer, pharmaceutically acceptable salt, ester, or prodrug thereof, wherein:
T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;
Q¹, Q², L¹ or L² are independently hydrogen, halogen, lower alkyl having from 1 to 6 carbon atoms, trifluoromethyl, or lower methoxy having from 1 to 6 carbon atoms; and
at least one of Q¹, Q², L¹ or L² is in the para position and is —S(O)ₙ—R, wherein n is 0, 1, or 2 and R is a lower alkyl radical having 1 to 6 carbon atoms, a lower haloalkyl radical having from 1 to 6 carbon atoms, or an —SO₂NH₂; or,
Q¹ and Q² are methylenedioxy; or
L¹ and L² are methylenedioxy; and
R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,
R²⁵ and R²⁶ are O; or,
R²⁷ and R²⁸ are O; or,
R²⁵, R²⁶, together with the carbon atom to which they are attached, form a saturated hydrocarbon ring having from 3 to 7 carbon atoms; or,
R²⁷, R²⁸, together with the carbon atom to which they are attached, form a saturated hydrocarbon ring having from 3 to 7 carbon atoms.

110. The composition of claim 109 wherein said Cox-2 inhibitor is N-(2-cyclohexyloxynitrophenyl)methane sulfonamide, or (E)-4-[(4-methylphenyl)(tetrahydro-2-oxo-3-furanylidene) methyl] benzenesulfonamide.

111. The composition of claim 109, wherein said Cox-2 inhibitor or isomer, pharmaceutically acceptable salt, ester, or prodrug thereof has a Cox-2 IC₅₀ of less than about 5 μmol/L.

112. The composition of claim 111, wherein said Cox-2 inhibitor or isomer, pharmaceutically acceptable salt, ester, or prodrug thereof has a selectivity ratio of Cox-2 inhibition to Cox-1 inhibition of at least about 1.5.

113. The composition of claim 112, wherein said Cox-2 inhibitor or isomer, pharmceutically acceptable salt, ester, or prodrug thereof has a Cox-2 IC₅₀ of less than about 1 μmol/L and a selectivity ratio of Cox-2 inhibition to Cox-1 inhibition of at least about 100.

114. The composition of claim 109, wherein said Cox-2 inhibitor or isomer, pharmaceutically acceptable salt, ester, or prodrug thereof has a Cox-1 IC₅₀ of at least about 1 μmol/L.

115. The composition of claim 114, wherein said Cox-2 inhibitor or isomer, pharmaceutically acceptable salt, ester, or prodrug thereof has a Cox-1 IC₅₀ of at least about 20 μmol/L.

116. The composition of claim 109, wherein said first amount is from about 0.001 to about 100 mg/day per kg of body weight of said subject and said second amount is from about 1 to about 600 mg/day per kg of body weight of said subject.

117. The composition of claim 116, wherein said first amount is from about 0.5 to about 50 mg/day per kg of body weight of said subject and said second amount is from about 100 to about 500 mg/day per kg of body weight of said subject.

118. The composition of claim 117, wherein said first amount is from about 1 to about 20 mg/day per kg of body weight of said subject and said second amount is from about 200 to about 400 mg/day per kg of body weight of said subject.

119. The composition of claim 109, wherein said subject is an animal.

120. The composition of claim 119, wherein said subject is a human.

121. The composition of claim 109, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered enterally or parenterally in one or more doses per day.

122. The composition of claim 109, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered substantially simultaneously.

123. The composition of claim 109, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered sequentially.

124. The composition of claim 109, wherein the neoplasia disorder is a tumor growth.

125. The composition of claim 124, wherein the tumor growth is a malignant tumor growth or a benign tumor growth.

126. The composition of claim 125, wherein the malignant tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

127. The composition of claim 125, wherein the malignant growth is a viral-related cancer.

128. The composition of claim 127, wherein the viral-related cancer includes cervical cancer, T-cell leukemia, lymphoma, and Kaposi's sarcoma.

129. The composition of claim 125, wherein the benign tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

130. The composition of claim 129, wherein the benign tumor growth is a fibroid tumor, an endometriosis, or a cyst.

131. A composition for treating neoplasia disorder comprising administering, to a subject in need thereof, a cyclooxygenase-2 (Cox-2) inhibitor in a first amount and a thalidomide, thalidomide analog, thalidomide hydrolysis product, thalidomide metabolite or thalidomide precursor in a second amount, wherein said first amount together with said second amount is a therapeutically effective amount of said Cox-2 inhibitor and said thalidomide, thalidomide analog, thalidomide hydrolysis product, thalidomide metabolite or thalidomide precursor, and wherein said Cox-2 inhibitor comprises B-1, B-2, B-3, B-4, B-5, B-6, B-7, B-8, B-9, B-10, B-11, B-12, B-13, B-14, B-15, B-16, B-17, B-18, B-19, B-20, B-21, B-22, B-23, B-24, B-25, B-26, B-27, B-28, B-29, B-30, B-31, B-32, B-33, B-34, B-35, B-36, B-37, B-38, B-39, B-40, B-41, B-42, B-43, B-44, B-45, B-46, B-47, B-48, B-49, B-50, B-51, B-52, B-53, B-54, B-55, B-56, B-57, B-58, B-59, B-60, B-61, B-62, B-63, B-64, B-65, B-66, B-67, B-68, B-69, B-70, B-71, B-72, B-73, B-74, B-75, B-76, B-77, B-78, B-79, B-80, B-81, B-82, B-83, B-84, B-85, B-86, B-87, B-88, B-89, B-90, B-91, B-92, B-93, B-94, B-95, B-96, B-97, B-98, B-99, B-100, B-101, B-102, B-103, B-104, B-105, B-106, B-107, B-108, B-109, B-110, B-111, B-112, B-113, B-114, B-115, B-116, B-117, B-118, B-119, B-120, B-121, B-122, B-123, B-124, B-125, B-126, B-127, B-128, B-129, B-130, B-131, B-132, B-133, B-134, B-135, B-136, B-137, B-138, B-139, B-140, B-141, B-142, B-143, B-144, B-145, B-146, B-147, B-148, B-149, B-150, B-151, B-152, B-153, B-154, B-155, B-156, B-157, B-158, B-159, B-160, B-161, B-162, B-163, B-164, B-165, B-166, B-167, B-168, B-169, B-170, B-171, B-172, B-173, B-174, B-175, B-176, B-177, B-178, B-179, B-180, B-181, B-182, B-183, B-184, B-185, B-186, B-187, B-188, B-189, B-190, B-191, B-192, B-193, B-194, B-195, B-196, B-197, B-198, B-199, B-200, B-201, B-202, B-203, B-204, B-205, B-206, B-207, B-208, B-209, B-210, B-211, B-212, B-213, B-214, B-215, B-216, B-217, B-218, B-219, B-220, B-221, B-222, B-223, B-224, B-225, B-226, B-227, B-228, B-229, B-230, B-231, B-232, or an isomer, a pharmaceutically acceptable salt, ester, or prodrug thereof, and,

132. The composition of claim 131 wherein said Cox-2 inhibitor is celecoxib (B-18), valdecoxib (B-19), deracoxib (B-20), rofecoxib (B-21), etoricoxib (B-22), JTE-522 (B-23), parecoxib (B-24), ABT-963 (B-25), or BMS-347070 (B-74), and an isomer, a pharmaceutically acceptable salt, ester, or prodrug thereof.

133. The composition of claim 132 wherein said Cox-2 inhibitor is celecoxib (B-18), rofecoxib (B-21), etoricoxib (B-22), JTE-522 (B-23), parecoxib (B-24), ABT-963 (B-25), or BMS-347070 (B-74).

134. The composition of claim 133, wherein said Cox-2 inhibitor is sodium parecoxib.

135. The composition of claim 131, wherein said Cox-2 inhibitor, isomer, pharmaceutically acceptable salt, ester, or prodrug thereof has a Cox-2 IC₅₀ of less than about 5 μmol/L.

136. The composition of claim 135, wherein said Cox-2 inhibitor, isomer, pharmaceutically acceptable salt, ester, or prodrug thereof has a selectivity ratio of Cox-2 inhibition to Cox-1 inhibition of at least about 1.5.

137. The composition of claim 136, wherein said Cox-2 inhibitor, isomer, pharmaceutically acceptable salt, ester, or prodrug thereof has a Cox-2 IC₅₀ of less than about 1 μmol/L and a selectivity ratio of Cox-2 inhibition to Cox-1 inhibition of at least about 100.

138. The composition of claim 131, wherein said Cox-2 inhibitor, isomer, pharmaceutically acceptable salt, ester, or prodrug thereof has a Cox- I IC₅₀ of at least about 1 μmol/L.

139. The composition of claim 138, wherein said Cox-2 inhibitor, isomer, pharmaceutically acceptable salt, ester, or prodrug thereof has a Cox-1 IC₅₀ of at least about 20 μmol/L.

140. The composition of claim 131, wherein said first amount is from about 0.001 to about 100 mg/day per kg of body weight of said subject and said second amount is from about 1 to about 600 mg/day per kg of body weight of said subject.

141. The composition of claim 140, wherein said first amount is from about 0.5 to about 50 mg/day per kg of body weight of said subject and said second amount is from about 100 to about 500 mg/day per kg of body weight of said subject.

142. The composition of claim 141, wherein said first amount is from about 1 to about 20 mg/day per kg of body weight of said subject and said second amount is from about 200 to about 400 mg/day per kg of body weight of said subject.

143. The composition of claim 131, wherein said subject is an animal.

144. The composition of claim 143, wherein said subject is a human.

145. The composition of claim 131, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered enterally or parenterally in one or more doses per day.

146. The composition of claim 131, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered substantially simultaneously.

147. The composition of claim 131, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered sequentially.

148. The composition of claim 131, wherein the neoplasia disorder is a tumor growth.

149. The composition of claim 148, wherein the tumor growth is a malignant tumor growth or a benign tumor growth.

150. The composition of claim 149, wherein the malignant tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

151. The composition of claim 149, wherein the malignant growth is a viral-related cancer.

152. The composition of claim 151, wherein the viral-related cancer includes cervical cancer, T-cell leukemia, lymphoma, and Kaposi's sarcoma.

153. The composition of claim 149, wherein the benign tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

154. The composition of claim 153, wherein the benign tumor growth is a fibroid tumor, an endometriosis, or a cyst.

155. A method for the treatment, prevention or inhibition of neoplasia disorder in a subject in need of such treatment comprising administering to the subject a cyclooxygenase-2 inhibitor or a pharmaceutically acceptable salt, ester or prodrug thereof in a first amount and a thalidomide, thalidomide analog, thalidomide hydrolysis product, thalidomide metabolite or thalidomide precursor in a second amount, wherein said first amount together with said second amount comprises a therapeutically effective amount for the treatment, prevention or inhibition of neoplasia disorder in said subject.

156. The method of claim 155, wherein said Cox-2 inhibitor or isomer, pharmaceutically acceptable salt, ester, or prodrug thereof has a Cox-2 IC₅₀ of less than about 5 μmol/L.

157. The method of claim 156, wherein said Cox-2 inhibitor or isomer, pharmaceutically acceptable salt, ester, or prodrug thereof has a selectivity ratio of Cox-2 inhibition to Cox-1 inhibition of at least about 1.5.

158. The method of claim 157, wherein said Cox-2 inhibitor or isomer, pharmceutically acceptable salt, ester, or prodrug thereof has a Cox-2 IC₅₀ of less than about 1 μmol/L and a selectivity ratio of Cox-2 inhibition to Cox-1 inhibition of at least about 100.

159. The method of claim 155, wherein said Cox-2 inhibitor or isomer, pharmaceutically acceptable salt, ester, or prodrug thereof has a Cox-1 IC₅₀ of at least about 1 μmol/L.

160. The method of claim 159, wherein said Cox-2 inhibitor or isomer, pharmaceutically acceptable salt, ester, or prodrug thereof has a Cox-1 IC₅₀ of at least about 20 μmol/L.

161. The method of claim 155, wherein said subject is an animal.

162. The method of claim 161, wherein said subject is a human.

163. The method of claim 155, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered enterally or parenterally in one or more doses per day.

164. The method of claim 155, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered substantially simultaneously.

165. The method of claim 155, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered sequentially.

166. The composition of claim 155, wherein the neoplasia disorder is a tumor growth.

167. The method of claim 166, wherein the tumor growth is a malignant tumor growth or a benign tumor growth.

168. The method of claim 167, wherein the malignant tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

169. The method of claim 167, wherein the malignant growth is a viral-related cancer.

170. The method of claim 169, wherein the viral-related cancer includes cervical cancer, T-cell leukemia, lymphoma, and Kaposi's sarcoma.

171. The method of claim 167, wherein the benign tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

172. The method of claim 171, wherein the benign tumor growth is a fibroid tumor, an endometriosis, or a cyst.

173. A method for the treatment, prevention or inhibition of neoplasia disorder in a subject in need of such treatment comprising administering to the subject a cyclooxygenase-2 inhibitor or a pharmaceutically acceptable salt, ester or prodrug thereof selected from the group consisting of substituted benzothiopyrans, dihydroquinolines, and dihydronaphtalenes in a first amount and a thalidomide, thalidomide analog, thalidomide hydrolysis product, thalidomide metabolite or thalidomide precursor in a second amount, wherein said first amount together with said second amount comprises a therapeutically effective amount for the treatment, prevention or inhibition of neoplasia disorder in said subject.

174. A method for treating neoplasia disorder comprising administering, to a subject in need thereof, a cyclooxygenase-2 (Cox-2) inhibitor in a first amount and a thalidomide, thalidomide analog, thalidomide hydrolysis product, thalidomide metabolite or thalidomide precursor in a second amount, wherein said first amount together with said second amount is a therapeutically effective amount of said Cox-2 inhibitor and said thalidomide, thalidomide analog, thalidomide hydrolysis product, thalidomide metabolite or thalidomide precursor, and wherein said Cox-2 inhibitor is represented by Formula (I): 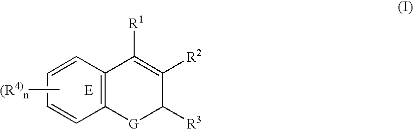
or an isomer, a pharmaceutically acceptable salt, ester, or prodrug thereof; wherein:
G is O, S or NR^{a};
R^{a} is alkyl;
R¹ is H or aryl;
R² is carboxyl, aminocarbonyl, alkylsulfonylaminocarbonyl or alkoxycarbonyl;
R³ is haloalkyl, alkyl, aralkyl, cycloalkyl or aryl optionally and independently substituted with one or more radicals selected from alkylthio, nitro and alkylsulfonyl;
n is an integer which is 1, 2, 3, or 4; and
each R⁴ is independently H, halo, alkyl, aryl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, mono- or dialkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, aralkylcarbonyl, heteroarylcarbonyl, arylcarbonyl, aminocarbonyl, alkylcarbonyl, aryl, or heteroaryl; wherein said aryl and heteroaryl radicals are optionally and independently substituted with one or more radicals which are alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy or alkylthio; or wherein R⁴ together with the atoms to which R⁴ is attached and the remainder of ring E forms a naphthyl radical.

175. The method of claim 174, wherein:
G is O or S;
R² is carboxyl, lower alkyl, lower aralkyl and lower alkoxycarbonyl;
R³ is lower haloalkyl, lower cycloalkyl and phenyl; and
each of one or more R⁴ is independently H, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, lower aralkylcarbonyl, lower alkylcarbonyl, and phenyl optionally and independently substituted with one or more radicals selected from the group consisting of alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy or alkylthio;
or wherein R⁴ together with the atoms to which R⁴ is attached and the remainder of ring E forms a naphthyl radical.

176. The method of claim 175, wherein:
R² is carboxyl;
R³ is lower haloalkyl; and
each of one or more R⁴ is independently H, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, or lower alkylcarbonyl;
or wherein R⁴ together with the atoms to which R⁴ is attached and the remainder of ring E forms a naphthyl radical.

177. The method of claim 176, wherein:
said lower haloalkyl R³ is fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, or trifluoromethyl; and each or one or more R⁴ is independently H, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, benzylaminosulfonyl, N-ethylsulfonyl, 2,2-dimethylethylaminosulfonyl, N,N-dimethylaminosulfonyl, isopropylaminosulfonyl, N-(2-methylpropyl)aminosulfonyl, N-morpholinosulfonyl, methylsulfonyl, benzylcarbonyl, 2,2-dimethylpropylcarbonyl, phenylacetyl, or phenyl;
or wherein R⁴ together with the atoms to which R⁴ is attached and the remainder of the ring E forms a naphthyl radical.

178. The method of claim 177, wherein:
R³ is trifluoromethyl or pentafluoroethyl; and each of one or more R⁴ is independently H, chloro, fluoro, bromo, iodo, methyl, ethyl, isopropyl, tert-butyl, methoxy, trifluoromethyl, trifluoromethoxy, N,N-diethylamino, N-phenylmethylaminosulfonyl, N-phenylethylaminosulfonyl, N-(2-furylmethyl)aminosulfonyl, N,N-dimethylaminosulfonyl, N-methylaminosulfonyl, benzylaminosulfonyl, N-(2,2-dimethylethyl)aminosulfonyl, isopropylaminosulfonyl, dimethylaminosulfonyl, 2-methylpropylaminosulfonyl, N-morpholinosulfonyl, methylsulfonyl, benzylcarbonyl, or phenyl;
or wherein R⁴ together with the atoms to which R⁴ is attached and the remainder of ring E forms a naphthyl radical.

179. The method of claim 178, wherein:
R³ is trifluoromethyl or pentafluoroethyl;
each of one or more R⁴ is independently H, methyl, ethyl, isopropyl, tert-butyl, chloro, bromo, fluoro, iodo, methyl, tert-butyl, trifluoromethoxy, methoxy, benzylcarbonyl, dimethylaminosulfonyl, isopropylaminosulfonyl, N-methylaminosulfonyl, benzylaminosulfonyl, phenylethylaminosulfonyl, methylpropylaminosulfonyl, methylsulfonyl, morpholinosulfonyl, N,N-diethylamino, or phenyl.

180. The method of claim 174, wherein said first amount is from about 0.001 to about 100 mg/day per kg of body weight of said subject and said second amount is from about 1 to about 600 mg/day per kg of body weight of said subject.

181. The method of claim 180, wherein said first amount is from about 0.5 to about 50 mg/day per kg of body weight of said subject and said second amount is from about 100 to about 500 mg/day per kg of body weight of said subject.

182. The method of claim 181, wherein said first amount is from about 1 to about 20 mg/day per kg of body weight of said subject and said second amount is from about 200 to about 400 mg/day per kg of body weight of said subject.

183. The method of claim 174, wherein said subject is an animal.

184. The method of claim 183, wherein said subject is a human.

185. The method of claim 174, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered enterally or parenterally in one or more doses per day.

186. The method of claim 174, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered substantially simultaneously.

187. The method of claim 174, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered sequentially.

188. The method of claim 174, wherein the neoplasia disorder is a tumor growth.

189. The composition of claim 188, wherein the tumor growth is a malignant tumor growth or a benign tumor growth.

190. The method of claim 189, wherein the malignant tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

191. The method of claim 189, wherein the malignant growth is a viral-related cancer.

192. The method of claim 191, wherein the viral-related cancer includes cervical cancer, T-cell leukemia, lymphoma, and Kaposi's sarcoma.

193. The method of claim 189, wherein the benign tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

194. The method of claim 193, wherein the benign tumor growth is a fibroid tumor, an endometriosis, or a cyst.

195. A method for the treatment, prevention or inhibition of neoplasia disorder in a subject in need of such treatment comprising administering to the subject a cyclooxygenase-2 inhibitor or a pharmaceutically acceptable salt, ester or prodrug thereof selected from the group consisting of tricylic Cox-2 inhibitors in a first amount and a thalidomide, thalidomide analog, thalidomide hydrolysis product, thalidomide metabolite or thalidomide precursor in a second amount, wherein said first amount together with said second amount comprises a therapeutically effective amount for the treatment, prevention or inhibition of neoplasia disorder in said subject.

196. A method for treating neoplasia disorder comprising administering, to a subject in need thereof, a cyclooxygenase-2 (Cox-2) inhibitor in a first amount and a thalidomide, thalidomide analog, thalidomide hydrolysis product, thalidomide metabolite or thalidomide precursor in a second amount, wherein said first amount together with said second amount is a therapeutically effective amount of said Cox-2 inhibitor and said thalidomide, thalidomide analog, thalidomide hydrolysis product, thalidomide metabolite or thalidomide precursor, and wherein said Cox-2 inhibitor is represented by Formula (II): 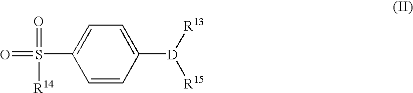
or an isomer, a pharmaceutically acceptable salt, ester, or prodrug thereof, wherein:
D is a partially unsaturated or saturated heterocyclyl ring or a partially unsaturated or saturated carbocyclic ring;
R¹³ is heterocyclyl, cycloalkyl, cycloalkenyl and aryl, wherein R¹³ is optionally substituted at a substitutable position with one or more radicals which are alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy or alkylthio;
R¹⁴ is methyl or amino; and
R¹⁵ is H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, or N-alkyl-N-arylaminosulfonyl.

197. The method of claim 196, wherein said Cox-2 inhibitor or isomer, pharmaceutically acceptable salt, ester, or prodrug thereof has a Cox-2 IC₅₀ of less than about 5 μmol/L.

198. The method of claim 197, wherein said Cox-2 inhibitor or isomer, pharmaceutically acceptable salt, ester, or prodrug thereof has a selectivity ratio of Cox-2 inhibition to Cox-1 inhibition of at least about 1.5.

199. The method of claim 198, wherein said Cox-2 inhibitor or isomer, pharmceutically acceptable salt, ester, or prodrug thereof has a Cox-2 IC₅₀ of less than about 1 μmol/L and a selectivity ratio of Cox-2 inhibition to Cox-1 inhibition of at least about 100.

200. The method of claim 196, wherein said Cox-2 inhibitor or isomer, pharmaceutically acceptable salt, ester, or prodrug thereof has a Cox- I IC₅₀ of at least about 1 μmol/L.

201. The method of claim 200, wherein said Cox-2 inhibitor or isomer, pharmaceutically acceptable salt, ester, or prodrug thereof has a Cox-1 IC₅₀ of at least about 20 μmol/L.

202. The method of claim 196, wherein said first amount is from about 0.001 to about 100 mg/day per kg of body weight of said subject and said second amount is from about 1 to about 600 mg/day per kg of body weight of said subject.

203. The method of claim 202, wherein said first amount is from about 0.5 to about 50 mg/day per kg of body weight of said subject and said second amount is from about 100 to about 500 mg/day per kg of body weight of said subject.

204. The method of claim 203, wherein said first amount is from about 1 to about 20 mg/day per kg of body weight of said subject and said second amount is from about 200 to about 400 mg/day per kg of body weight of said subject.

205. The method of claim 196, wherein said subject is an animal.

206. The method of claim 205, wherein said subject is a human.

207. The method of claim 196, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered enterally or parenterally in one or more doses per day.

208. The method of claim 196, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered substantially simultaneously.

209. The method of claim 196, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered sequentially.

210. The method of claim 196, wherein the neoplasia disorder is a tumor growth.

211. The method of claim 210, wherein the tumor growth is a malignant tumor growth or a benign tumor growth.

212. The method of claim 211, wherein the malignant tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

213. The method of claim 211, wherein the malignant growth is a viral-related cancer.

214. The method of claim 213, wherein the viral-related cancer includes cervical cancer, T-cell leukemia, lymphoma, and Kaposi's sarcoma.

215. The method of claim 211, wherein the benign tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

216. The method of claim 215, wherein the benign tumor growth is a fibroid tumor, an endometriosis, or a cyst.

217. A method for the treatment, prevention or inhibition of neoplasia disorder in a subject in need of such treatment comprising administering to the subject a cyclooxygenase-2 inhibitor or a pharmaceutically acceptable salt, ester or prodrug thereof selected from the group consisting of phenylacetic acid derivatives in a first amount and a thalidomide, thalidomide analog, thalidomide hydrolysis product, thalidomide metabolite or thalidomide precursor in a second amount, wherein said first amount together with said second amount comprises a therapeutically effective amount for the treatment, prevention or inhibition of neoplasia disorder in said subject.

218. A method for treating neoplasia disorder comprising administering, to a subject in need thereof, a cyclooxygenase-2 (Cox-2) inhibitor in a first amount and a thalidomide, thalidomide analog, thalidomide hydrolysis product, thalidomide metabolite or thalidomide precursor in a second amount, wherein said first amount together with said second amount is a therapeutically effective amount of said Cox-2 inhibitor and said thalidomide, thalidomide analog, thalidomide hydrolysis product, thalidomide metabolite or thalidomide precursor, and wherein said Cox-2 inhibitor is represented by Formula (III): 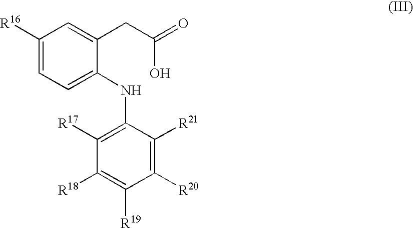
or an isomer, a pharmaceutically acceptable salt, ester, or prodrug thereof, wherein:
R¹⁶ is methyl or ethyl;
R¹⁷ is chloro or fluoro;
R¹⁸ is hydrogen or fluoro;
R¹⁹ is hydrogen, fluoro, chloro, methyl, ethyl, methoxy, ethoxy or hydroxy;
R²⁰ is hydrogen or fluoro; and
R²¹ is chloro, fluoro, trifluoromethyl or methyl, provided that R¹⁷, R¹⁸, R¹⁹ and R²⁰ are not all fluoro when R¹⁶ is ethyl and R¹⁹ is H.

219. The method of claim 218, wherein:
R¹⁶ is ethyl; R¹⁷ and R¹⁹ are chloro; R¹⁸ and R²⁰ are hydrogen; and R²¹ is methyl.

220. The method of claim 218, wherein said Cox-2 inhibitor or isomer, pharmaceutically acceptable salt, ester, or prodrug thereof has a Cox-2 IC₅₀ of less than about 5 μmol/L.

221. The method of claim 220, wherein said Cox-2 inhibitor or isomer, pharmaceutically acceptable salt, ester, or prodrug thereof has a selectivity ratio of Cox-2 inhibition to Cox-1 inhibition of at least about 1.5.

222. The method of claim 221, wherein said Cox-2 inhibitor or isomer, pharmceutically acceptable salt, ester, or prodrug thereof has a Cox-2 IC₅₀ of less than about 1 μmol/L and a selectivity ratio of Cox-2 inhibition to Cox-1 inhibition of at least about 100.

223. The method of claim 218, wherein said Cox-2 inhibitor or isomer, pharmaceutically acceptable salt, ester, or prodrug thereof has a Cox-1 IC₅₀ of at least about 1 μmol/L.

224. The method of claim 223, wherein said Cox-2 inhibitor or isomer, pharmaceutically acceptable salt, ester, or prodrug thereof has a Cox-1 IC₅₀ of at least about 20 μmol/L.

225. The method of claim 218, wherein said first amount is from about 0.001 to about 100 mg/day per kg of body weight of said subject and said second amount is from about 1 to about 600 mg/day per kg of body weight of said subject.

226. The method of claim 225, wherein said first amount is from about 0.5 to about 50 mg/day per kg of body weight of said subject and said second amount is from about 100 to about 500 mg/day per kg of body weight of said subject.

227. The method of claim 226, wherein said first amount is from about 1 to about 20 mg/day per kg of body weight of said subject and said second amount is from about 200 to about 400 mg/day per kg of body weight of said subject.

228. The method of claim 218, wherein said subject is an animal.

229. The method of claim 228, wherein said subject is a human.

230. The method of claim 218, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered enterally or parenterally in one or more doses per day.

231. The method of claim 218, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered substantially simultaneously.

232. The method of claim 218, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered sequentially.

233. The composition of claim 218, wherein the neoplasia disorder is a tumor growth.

234. The composition of claim 233, wherein the tumor growth is a malignant tumor growth or a benign tumor growth.

235. The method of claim 234, wherein the malignant tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

236. The method of claim 234, wherein the malignant growth is a viral-related cancer.

237. The method of claim 236, wherein the viral-related cancer includes cervical cancer, T-cell leukemia, lymphoma, and Kaposi's sarcoma.

238. The method of claim 234, wherein the benign tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

239. The method of claim 238, wherein the benign tumor growth is a fibroid tumor, an endometriosis, or a cyst.

240. A method for treating neoplasia disorder comprising administering, to a subject in need thereof, a cyclooxygenase-2 (Cox-2) inhibitor in a first amount and a thalidomide, thalidomide analog, thalidomide hydrolysis product, thalidomide metabolite or thalidomide precursor in a second amount, wherein said first amount together with said second amount is a therapeutically effective amount of said Cox-2 inhibitor and said thalidomide, thalidomide analog, thalidomide hydrolysis product, thalidomide metabolite or thalidomide precursor, and wherein said Cox-2 inhibitor is represented by Formula (IV): 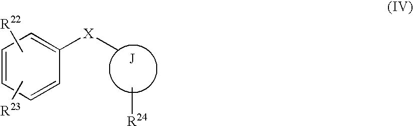
or an isomer, a pharmaceutically acceptable salt, ester, or prodrug thereof, wherein:
X is O or S;
J is a carbocycle or a heterocycle;
R²² is NHSO₂CH₃ or F;
R²³ is H, NO₂, or F; and
R²⁴ is H, NHSO₂CH₃, or (SO₂CH₃)C₆H₄.

241. The method of claim 240 wherein said Cox-2 inhibitor is nimesulide (B-212), flosulide (B-213), NS-398 (B-26), L-745337 (B-214), RWJ-63556 (B-215), or L-784512 (B-216).

242. The method of claim 240, wherein said Cox-2 inhibitor or isomer, pharmaceutically acceptable salt, ester, or prodrug thereof has a Cox-2 IC₅₀ of less than about 5 μmol/L.

243. The method of claim 242, wherein said Cox-2 inhibitor or isomer, pharmaceutically acceptable salt, ester, or prodrug thereof has a selectivity ratio of Cox-2 inhibition to Cox-1 inhibition of at least about 1.5.

244. The method of claim 243, wherein said Cox-2 inhibitor or isomer, pharmceutically acceptable salt, ester, or prodrug thereof has a Cox-2 IC₅₀ of less than about 1 μmol/L and a selectivity ratio of Cox-2 inhibition to Cox-1 inhibition of at least about 100.

245. The method of claim 240, wherein said Cox-2 inhibitor or isomer, pharmaceutically acceptable salt, ester, or prodrug thereof has a Cox-1 IC₅₀ of at least about 1 μmol/L.

246. The method of claim 245, wherein said Cox-2 inhibitor or isomer, pharmaceutically acceptable salt, ester, or prodrug thereof has a Cox-1 IC₅₀ of at least about 20 μmol/L.

247. The method of claim 240, wherein said first amount is from about 0.001 to about 100 mg/day per kg of body weight of said subject and said second amount is from about 1 to about 600 mg/day per kg of body weight of said subject.

248. The method of claim 247, wherein said first amount is from about 0.5 to about 50 mg/day per kg of body weight of said subject and said second amount is from about 100 to about 500 mg/day per kg of body weight of said subject.

249. The method of claim 248, wherein said first amount is from about 1 to about 20 mg/day per kg of body weight of said subject and said second amount is from about 200 to about 400 mg/day per kg of body weight of said subject.

250. The method of claim 240, wherein said subject is an animal.

251. The method of claim 250, wherein said subject is a human.

252. The method of claim 240, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered enterally or parenterally in one or more doses per day.

253. The method of claim 240, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered substantially simultaneously.

254. The method of claim 240, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered sequentially.

255. The method of claim 240, wherein the neoplasia disorder is a tumor growth.

256. The method of claim 255, wherein the tumor growth is a malignant tumor growth or a benign tumor growth.

257. The method of claim 256, wherein the malignant tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

258. The method of claim 256, wherein the malignant growth is a viral-related cancer.

259. The method of claim 258, wherein the viral-related cancer includes cervical cancer, T-cell leukemia, lymphoma, and Kaposi's sarcoma.

260. The method of claim 256, wherein the benign tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

261. The method of claim 260, wherein the benign tumor growth is a fibroid tumor, an endometriosis, or a cyst.

262. A method for the treatment, prevention or inhibition of neoplasia disorder in a subject in need of such treatment comprising administering to the subject a cyclooxygenase-2 inhibitor or a pharmaceutically acceptable salt, ester or prodrug thereof selected from the group consisting of diarylmethyldenefuran and diarylmethyldenefuran derivatives in a first amount and a thalidomide, thalidomide analog, thalidomide hydrolysis product, thalidomide metabolite or thalidomide precursor in a second amount, wherein said first amount together with said second amount comprises a therapeutically effective amount for the treatment, prevention or inhibition of neoplasia disorder in said subject.

263. A method for treating neoplasia disorder comprising administering, to a subject in need thereof, a cyclooxygenase-2 (Cox-2) inhibitor in a first amount and a thalidomide, thalidomide analog, thalidomide hydrolysis product, thalidomide metabolite or thalidomide precursor in a second amount, wherein said first amount together with said second amount is a therapeutically effective amount of said Cox-2 inhibitor and said thalidomide, thalidomide analog, thalidomide hydrolysis product, thalidomide metabolite or thalidomide precursor, and wherein said Cox-2 inhibitor is represented by Formula (V): 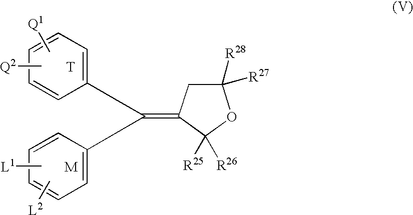
or an isomer, pharmaceutically acceptable salt, ester, or prodrug thereof, wherein:
T and M independently are phenyl, naphthyl, a radical derived from a heterocycle comprising 5 to 6 members and possessing from 1 to 4 heterotoms, or a radical derived from a saturated hydrocarbon ring having from 3 to 7 carbon atoms;
Q¹, Q², L¹ or L² are independently hydrogen, halogen, lower alkyl having from 1 to 6 carbon atoms, trifluoromethyl, or lower methoxy having from 1 to 6 carbon atoms; and
at least one of Q¹, Q², L¹ or L² is in the para position and is —S(O)ₙ—R, wherein n is 0, 1, or 2 and R is a lower alkyl radical having 1 to 6 carbon atoms, a lower halo alkyl 20 radical having from 1 to 6 carbon atoms, or an —SO₂NH₂; or,
Q¹ and Q² are methylenedioxy; or
L¹ and L² are methylenedioxy; and
R²⁵, R²⁶, R²⁷, and R²⁸ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,
R²⁵ and R²⁶ are O; or,
R²⁷ and R²⁸ are O; or,
R²⁵, R²⁶, together with the carbon atom to which they are attached, form a saturated hydrocarbon ring having from 3 to 7 carbon atoms; or,
R²⁷, R²⁸, together with the carbon atom to which they are attached, form a saturated hydrocarbon ring having from 3 to 7 carbon atoms.

264. The method of claim 263 wherein said Cox-2 inhibitor is N-(2-cyclohexyloxynitrophenyl)methane sulfonamide, or (E)-4-[(4-methylphenyl)(tetrahydro-2-oxo-3-furanylidene) methyl] benzenesulfonamide.

265. The method of claim 263, wherein said Cox-2 inhibitor or isomer, pharmaceutically acceptable salt, ester, or prodrug thereof has a Cox-2 IC₅₀ of less than about 5 μmol/L.

266. The method of claim 265, wherein said Cox-2 inhibitor or isomer, pharmaceutically acceptable salt, ester, or prodrug thereof has a selectivity ratio of Cox-2 inhibition to Cox-1 inhibition of at least about 1.5.

267. The method of claim 266, wherein said Cox-2 inhibitor or isomer, pharmceutically acceptable salt, ester, or prodrug thereof has a Cox-2 IC₅₀ of less than about 1 μmol/L and a selectivity ratio of Cox-2 inhibition to Cox-1 inhibition of at least about 100.

268. The method of claim 263, wherein said Cox-2 inhibitor or isomer, pharmaceutically acceptable salt, ester, or prodrug thereof has a Cox-1 IC₅₀ of at least about 1 μmol/L.

269. The method of claim 268, wherein said Cox-2 inhibitor or isomer, pharmaceutically acceptable salt, ester, or prodrug thereof has a Cox-1 IC₅₀ of at least about 20 μmol/L.

270. The method of claim 263, wherein said first amount is from about 0.001 to about 100 mg/day per kg of body weight of said subject and said second amount is from about 1 to about 600 mg/day per kg of body weight of said subject.

271. The method of claim 270, wherein said first amount is from about 0.5 to about 50 mg/day per kg of body weight of said subject and said second amount is from about 100 to about 500 mg/day per kg of body weight of said subject.

272. The method of claim 271, wherein said first amount is from about 1 to about 20 mg/day per kg of body weight of said subject and said second amount is from about 200 to about 400 mg/day per kg of body weight of said subject.

273. The method of claim 263, wherein said subject is an animal.

274. The method of claim 273, wherein said subject is a human.

275. The method of claim 263, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered enterally or parenterally in one or more doses per day.

276. The method of claim 263, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered substantially simultaneously.

277. The method of claim 263, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered sequentially.

278. The method of claim 263, wherein the neoplasia disorder is a tumor growth.

279. The method of claim 278, wherein the tumor growth is a malignant tumor growth or a benign tumor growth.

280. The method of claim 279, wherein the malignant tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

281. The method of claim 279, wherein the malignant growth is a viral-related cancer.

282. The method of claim 281, wherein the viral-related cancer includes cervical cancer, T-cell leukemia, lymphoma, and Kaposi's sarcoma.

283. The method of claim 279, wherein the benign tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

284. The method of claim 283, wherein the benign tumor growth is a fibroid tumor, an endometriosis, or a cyst.

285. A method for treating neoplasia disorder comprising administering, to a subject in need thereof, a cyclooxygenase-2 (Cox-2) inhibitor in a first amount and a thalidomide, thalidomide analog, thalidomide hydrolysis product, thalidomide metabolite or thalidomide precursor in a second amount, wherein said first amount together with said second amount is a therapeutically effective amount of said Cox-2 inhibitor and said thalidomide, thalidomide analog, thalidomide hydrolysis product, thalidomide metabolite or thalidomide precursor, and wherein said Cox-2 inhibitor comprises B-1, B-2, B-3, B-4, B-5, B-6, B-7, B-8, B-9, B-10, B-11, B-12, B-13, B-14, B-15, B-16, B-17, B-18, B-19, B-20, B-21, B-22, B-23, B-24, B-25, B-26, B-27, B-28, B-29, B-30, B-31, B-32, B-33, B-34, B-35, B-36, B-37, B-38, B-39, B-40, B-41, B-42, B-43, B-44, B-45, B-46, B-47, B-48, B-49, B-50, B-51, B-52, B-53, B-54, B-55, B-56, B-57, B-58, B-59, B-60, B-61, B-62, B-63, B-64, B-65, B-66, B-67, B-68, B-69, B-70, B-71, B-72, B-73, B-74, B-75, B-76, B-77, B-78, B-79, B-80, B-81, B-82, B-83, B-84, B-85, B-86, B-87, B-88, B-89, B-90, B-91, B-92, B-93, B-94, B-95, B-96, B-97, B-98, B-99, B-100, B-101, B-102, B-103, B-104, B-105, B-106, B-107, B-108, B-109, B-110, B-111, B-112, B-113, B-114, B-115, B-116, B-117, B-118, B-119, B-120, B-121, B-122, B-123, B-124, B-125, B-126, B-127, B-128, B-129, B-130, B-131, B-132, B-133, B-134, B-135, B-136, B-137, B-138, B-139, B-140, B-141, B-142, B-143, B-144, B-145, B-146, B-147, B-148, B-149, B-150, B-151, B-152, B-153, B-154, B-155, B-156, B-157, B-158, B-159, B-160, B-161, B-162, B-163, B-164, B-165, B-166, B-167, B-168, B-169, B-170, B-171, B-172, B-173, B-174, B-175, B-176, B-177, B-178, B-179, B-180, B-181, B-182, B-183, B-184, B-185, B-186, B-187, B-188, B-189, B-190, B-191, B-192, B-193, B-194, B-195, B-196, B-197, B-198, B-199, B-200, B-201, B-202, B-203, B-204, B-205, B-206, B-207, B-208, B-209, B-210, B-211, B-212, B-213, B-214, B-215, B-216, B-217, B-218, B-219, B-220, B-221, B-222, B-223, B-224, B-225, B-226, B-227, B-228, B-229, B-230, B-231, B-232, or an isomer, a pharmaceutically acceptable salt, ester, or prodrug thereof.

286. The method of claim 285 wherein said Cox-2 inhibitor is celecoxib (B-18), valdecoxib (B-19), deracoxib (B-20), rofecoxib (B-21), etoricoxib (B-22), JTE-522 (B-23), parecoxib (B-24), ABT-963 (B-25), or BMS-347070 (B-74), and an isomer, a pharmaceutically acceptable salt, ester, or prodrug thereof.

287. The method of claim 286 wherein said Cox-2 inhibitor is celecoxib (B-18), rofecoxib (B-21), etoricoxib (B-22), JTE-522 (B-23), parecoxib (B-24), ABT-963 (B-25), or BMS-347070 (B-74).

288. The method of claim 287, wherein said Cox-2 inhibitor is sodium parecoxib.

289. The method of claim 285, wherein said Cox-2 inhibitor, isomer, pharmaceutically acceptable salt, ester, or prodrug thereof has a Cox-2 IC₅₀ of less than about 5 μmol/L.

290. The method of claim 289, wherein said Cox-2 inhibitor, isomer, pharmaceutically acceptable salt, ester, or prodrug thereof has a selectivity ratio of Cox-2 inhibition to Cox-1 inhibition of at least about 1.5.

291. The method of claim 290, wherein said Cox-2 inhibitor, isomer, pharmaceutically acceptable salt, ester, or prodrug thereof has a Cox-2 IC₅₀ of less than about 1 μmol/L and a selectivity ratio of Cox-2 inhibition to Cox-1 inhibition of at least about 100.

292. The method of claim 285, wherein said Cox-2 inhibitor, isomer, pharmaceutically acceptable salt, ester, or prodrug thereof has a Cox-1 IC₅₀ of at least about 1 μmol/L.

293. The method of claim 292, wherein said Cox-2 inhibitor, isomer, pharmaceutically acceptable salt, ester, or prodrug thereof has a Cox-1 IC₅₀ of at least about 20 μmol/L.

294. The method of claim 293, wherein said first amount is from about 0.001 to about 100 mg/day per kg of body weight of said subject and said second amount is from about 1 to about 600 mg/day per kg of body weight of said subject.

295. The method of claim 294, wherein said first amount is from about 0.5 to about 50 mg/day per kg of body weight of said subject and said second amount is from about 100 to about 500 mg/day per kg of body weight of said subject.

296. The method of claim 295, wherein said first amount is from about 1 to about 20 mg/day per kg of body weight of said subject and said second amount is from about 200 to about 400 mg/day per kg of body weight of said subject.

297. The method of claim 285, wherein said subject is an animal.

298. The method of claim 297, wherein said subject is a human.

299. The method of claim 285, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered enterally or parenterally in one or more doses per day.

300. The method of claim 285, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered substantially simultaneously.

301. The method of claim 285, wherein said Cox-2 inhibitor or isomer, pharmaceutically acceptable salt, ester, or prodrug thereof and said thalidomide, thalidomide analog, thalidomide hydrolysis product, thalidomide metabolite or thalidomide precursor are administered sequentially.

302. The method of claim 285, wherein the neoplasia disorder is a tumor growth.

303. The method of claim 302, wherein the tumor growth is a malignant tumor growth or a benign tumor growth.

304. The composition of claim 303, wherein the malignant tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

305. The method of claim 303, wherein the malignant growth is a viral-related cancer.

306. The method of claim 305, wherein the viral-related cancer includes cervical cancer, T-cell leukemia, lymphoma, and Kaposi's sarcoma.

307. The method of claim 303, wherein the benign tumor growth is in a location selected from the group consisting of the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, and dermis.

308. The method of claim 307, wherein the benign tumor growth is a fibroid tumor, an endometriosis, or a cyst.

309. A method of inhibiting angiogenesis, said method comprising administering a composition comprising a cyclooxygenase-2 inhibitor or a pharmaceutically acceptable salt, ester or prodrug thereof in a first amount and a thalidomide, thalidomide analog, thalidomide hydrolysis product, thalidomide metabolite or thalidomide precursor in a second amount, wherein said first amount together with said second amount comprises a therapeutically effective amount for the treatment, prevention or inhibition of angiogenesis.
